(19) United States
(12) Patent Application Publication
(10) Pub. No.: US 2016/0311785 A1
(43) Pub. Date: Oct. 27, 2016

(54) TRIAZINE MEDIATED LIVING RADICAL CONTROLLED POLYMERIZATION

(71) Applicants: DOW GLOBAL TECHNOLOGIES LLC, Midland, MI (US); THE REGENTS OF THE UNIVERSITY OF CALIFORNIA, Oakland, CA (US)

(72) Inventors: Jetsuda AREEPHONG, Goleta, CA (US); Nicolas TREAT, Goleta, CA (US); John W. KRAMER, Midland, MI (US); Matthew D. CHRISTIANSON, Midland, MI (US); Hazel A. COLLINS, Santa Barbara, CA (US)

(21) Appl. No.: 15/030,434

(22) PCT Filed: Oct. 20, 2014
(86) PCT No.: PCT/US2014/061292

§ 371 Date: Apr. 19, 2016

Related U.S. Application Data
(60) Provisional application No. 61/893,481, filed on Oct. 21, 2013.

(51) Int. Cl. C07D 253/10; C08F 12/08; C07D 471/04

(52) U.S. Cl. CPC C07D 253/10; C07D 471/04; C08F 12/08

(57) ABSTRACT

The disclosure provides modular triazine-based unimolecular initiator compounds useful in controlled radical polymerizations of vinyl-containing monomers.

### CROSS-REFERENCE TO RELATED APPLICATION

This application claims priority to U.S. Provisional Application No. 61/893,481, filed Oct. 21, 2013, the disclosure of which is hereby incorporated by reference in its entirety.

### BACKGROUND OF THE INVENTION

1. Field of the Invention

The disclosure provides modular triazine-based unimolecular initiator compounds useful in controlled radical polymerizations of vinyl-containing monomers.

2. Description of Related Art

Controlled radical polymerizations (CRP) provide well-defined polymers with complex architectures and rich functionality that are critical to many state-of-the-art applications. Three techniques dominate due to their simplicity and functional group tolerance: atom transfer radical polymerization (ATRP), reversible addition-fragmentation chain transfer polymerization (RAFT), and nitroxide mediated polymerization (NMP). These methods mimic ionic polymerization in their ability to produce targeted molecular weights and low molecular weight distributions, and also offer wide monomer tolerance. NMP is often preferred in applications where the potential metal and sulfur contamination inherent to ATRP and RAFT is a concern (i.e. block copolymer lithography, microelectronics, etc.). Key to NMPs mechanism is a stable nitroxide radical that reversibly caps the growing chain end. However, homopolymerization of methacrylates in NMP is limited to uniquely designed unimers which are unable to control the polymerization of styrene. A number of other persistent radicals have been employed as mediating species for polymerization, including (arylazo)oxy, borinate, triazolinyl, and verdazyl. One such radical is benzo-1,2,4-triazinyl (triazine) radical first reported in 1968 (H. M. Blatter, H. Lukaszewski, Tetrahedron Lett. 1968, 9, 2701-2705), which is highly stable in air. Use of benzo-1,2,4-triazinyl (triazine) radical was reported in the synthesis of low polydispersity polystyrene (PDI<1.2) (Demetriou, et al., Polym. Int. 2013, DOI: 10.1002/pi.4566.) However, linear growth in molecular weight with conversion was not observed. Additionally, theoretical and experimental molecular weights were not in agreement, indicating a lack of control still present in the system despite low polydispersities.

### SUMMARY OF THE INVENTION

In a broad aspect, the disclosure provides modular triazine-based unimolecular initiator compounds useful in controlled radical polymerizations. Unexpectedly, the compounds and methods of the disclosure showed control of homopolymerization, and targeted molecular weights and narrow molecular weight distributions were achieved. The compounds and methods of the disclosure also showed control of random copolymerizations, for example, of styrene with butyl acrylate and methyl methacrylate.

Thus, one aspect of the disclosure provides compound of formula I:

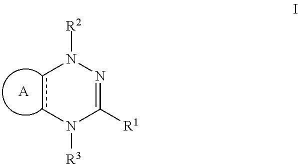

or an acceptable salt thereof, wherein
the dashed line represents an optional double bond;
A is selected from cycloalkyl, aryl, heteroaryl, and heterocyclyl, each of which is independently optionally substituted with one or more R⁴;
   wherein each R⁴ is independently selected from the group consisting of halogen, —NO₂, —CN, C₁-C₂₀ alkyl, C₂-C₂₀ alkenyl, C₂-C₂₀ alkynyl optionally substituted with —Si(C₁-C₆ alkyl)₃, C₁-C₂₀ haloalkyl, —OH, C₁-C₂₀ alkoxy, C₁-C₂₀ haloalkoxy, hydroxy(C₁-C₂₀ alkyl), alkoxy(C₁-C₂₀ alkyl), —NH₂, —NH(C₁-C₂₀ alkyl), —N(C₁-C₂₀ alkyl)₂, —CONH₂, —CONH(C₁-C₂₀ alkyl), —CON(C₁-C₂₀ alkyl)₂, —NHCO(C₁-C₂₀ alkyl), —NHCO(C₁-C₂₀ alkoxy), —N(C₁-C₂₀ alkyl)CO(C₁-C₂₀ alkyl), —CO₂H, —CO₂(C₁-C₂₀ alkyl), —OCO(C₁-C₂₀ alkyl), —CO₂(aryl), —S(O)₀₋₂—(C₁-C₂₀ alkyl), —S(O)₀₋₂-aryl, —S(O)₀₋₂-heteroaryl, —P(O)(OH)₂, —P(O)(C₁-C₂₀ alkoxy)₂, —P(O)(aryloxy)₂, cycloalkyl, cycloalkyl(C₁-C₂₀ alkyl), aryl, aryl(C₁-C₂₀ alkyl), heteroaryl, heteroaryl(C₁-C₂₀ alkyl), heterocyclyl, and heterocyclyl(C₁-C₂₀ alkyl), or two R⁴ groups on the same non-aromatic atom form an oxo;
R¹ is C₁-C₂₀ alkyl, C₂-C₂₀ alkenyl, C₂-C₂₀ alkynyl optionally substituted with —Si(C₁-C₆ alkyl)₃, C₄-C₁₀ haloalkyl, —CO₂(C₁-C₂₀ alkyl), —S(O)₀₋₂—(C₁-C₂₀ alkyl), —S(O)₀₋₂-aryl, —S(O)₀₋₂-heteroaryl, —P(O)(OH)₂, —P(O)(C₁-C₂₀ alkoxy)₂, —P(O)(aryloxy)₂, aryl, aryl(C₁-C₂₀ alkyl), heteroaryl, or heteroaryl(C₁-C₂₀ alkyl), wherein each of which is independently optionally substituted with one or more R⁵;
R² is C₁-C₂₀ alkyl, C₂-C₂₀ alkenyl, C₂-C₂₀ alkynyl optionally substituted with —Si(C₁-C₆ alkyl)₃, C₄-C₁₀ haloalkyl, —CO₂(C₁-C₂₀ alkyl), —S(O)₀₋₂—(C₁-C₂₀ alkyl), —S(O)₀₋₂-aryl, —S(O)₀₋₂-heteroaryl, —P(O)(OH)₂, —P(O)(C₁-C₂₀ alkoxy)₂, —P(O)(aryloxy)₂, aryl, aryl(C₁-C₂₀ alkyl), heteroaryl, or heteroaryl(C₁-C₂₀ alkyl), wherein each of which is independently optionally substituted with one or more R⁶;
   wherein each R⁵ and R⁶ are independently selected from the group consisting of halogen, —NO₂, —CN, C₁-C₂₀ alkyl, C₂-C₂₀ alkenyl, C₂-C₂₀ alkynyl optionally substituted with —Si(C₁-C₆ alkyl)₃, C₁-C₂₀ haloalkyl, —OH, C₁-C₂₀ alkoxy, C₁-C₂₀ haloalkoxy, hydroxy(C₁-C₂₀ alkyl), alkoxy(C₁-C₂₀ alkyl), —NH₂, —NH(C₁-C₂₀ alkyl), —N(C₁-C₂₀ alkyl)₂, —CONH₂, —CONH(C₁-C₂₀ alkyl), —CON(C₁-C₂₀ alkyl)₂, —NHCO(C₁-C₂₀ alkyl), —N(C₁-C₂₀ alkyl)CO(C₁-C₂₀ alkyl), —CO₂H, —CO₂(C₁-C₂₀ alkyl), —OCO(C₁-C₂₀ alkyl), —CO₂(aryl), —S(O)₀₋₂—(C₁-C₂₀ alkyl), —S(O)₀₋₂-aryl, —S(O)₀₋₂-heteroaryl, —P(O)(OH)₂, —P(O)(C₁-C₂₀ alkoxy)₂, —P(O)(aryloxy)₂, cycloalkyl, cycloalkyl(C₁-C₂₀ alkyl), aryl, aryl(C₁-C₂₀ alkyl), heteroaryl, heteroaryl(C₁-C₂₀ alkyl), heterocyclyl, and heterocyclyl(C₁-C₂₀ alkyl), or two R⁵ groups on the same non-aromatic atom form an oxo, or two R⁶ groups on the same non-aromatic atom form an oxo; and
R³ is

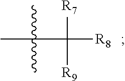

R⁷ is hydrogen, C₁-C₂₀ alkyl, or aryl, wherein alkyl or aryl moiety is optionally substituted with one or more R¹¹;
R⁸ is hydrogen, C₁-C₂₀ alkyl, aryl, —CO₂R¹⁰, or —CON(R¹⁰)₂; and
R⁹ is C₄-C₂₀ alkyl, aryl, aryl(C₁-C₂₀ alkyl), heteroaryl, heteroaryl(C₁-C₂₀ alkyl), heterocyclyl, heterocyclyl(C₁-C₂₀ alkyl), —CO₂R¹⁰, —CON(R¹⁰)₂, or —CN, wherein each alkyl, aryl, heteroaryl, or heterocyclyl moiety is optionally substituted with one or more R¹¹; and
wherein each R¹⁰ is independently selected from the group consisting of hydrogen, C₁-C₂₀ alkyl, or aryl, wherein each alkyl or aryl moiety is optionally substituted with one or more R¹¹;
wherein each R¹¹ is independently selected from the group consisting of halogen, —NO₂, —CN, C₁-C₂₀ alkyl, C₂-C₂₀ alkenyl, C₂-C₂₀ alkynyl optionally substituted with —Si(C₁-C₆ alkyl)₃, C₁-C₂₀ haloalkyl, —OH, C₁-C₂₀ alkoxy, C₁-C₂₀ haloalkoxy, hydroxy(C₁-C₂₀ alkyl), alkoxy(C₁-C₂₀ alkyl), —NH₂, —NH(C₁-C₂₀ alkyl), —N(C₁-C₂₀ alkyl)₂, —CONH₂, —CONH(C₁-C₂₀ alkyl), —CON(C₁-C₂₀ alkyl)₂, —NHCO(C₁-C₂₀ alkyl), —N(C₁-C₂₀ alkyl)CO(C₁-C₂₀ alkyl), amino(C₁-C₂₀ alkyl), —CO₂H, —CO₂(C₁-C₂₀ alkyl), —OCO(C₁-C₂₀ alkyl), —CO₂(aryl), —S(O)₀₋₂—(C₁-C₂₀ alkyl), —S(O)₀₋₂-aryl, and —S(O)₀₋₂-heteroaryl, or two R¹¹ that are on non-aromatic atom form an oxo;
or R³ is a polymeric group resulting from polymerization of one or more of vinyl-containing monomers.

The disclosure also provides compounds of formula I, wherein the dashed line is a double bond, and is of formula II:

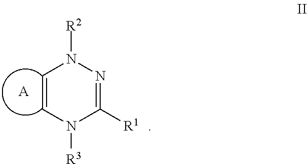

The disclosure also provides synthetic intermediates that are useful in making the compounds of formula I or II.

The disclosure also provides methods of preparing compounds of the disclosure and the intermediates used in those methods.

Another aspect of the disclosure provides methods for polymerizing one or more vinyl-containing monomers comprising contacting one or more vinyl-containing monomers with one or more compounds of formula I or II.

In another aspect, the methods of the disclosure provide radical-mediated polymerization of one or more vinyl-containing monomers comprising contacting one or more vinyl-containing monomers with one or more compounds of formula I or II.

In yet another aspect, the methods of the disclosure provide controlled radical-mediated polymerization of one or more vinyl-containing monomers comprising contacting one or more vinyl-containing monomers with one or more compounds of formula I or II.

### DETAILED DESCRIPTION OF THE INVENTION

The disclosure also provides compounds of formula I or II, wherein
A is selected from aryl and heteroaryl, each of which is independently optionally substituted with one or more R⁴;
   wherein each R⁴ is independently selected from the group consisting of halogen, —NO₂, —CN, C₁-C₂₀ alkyl, C₂-C₂₀ alkenyl, C₂-C₂₀ alkynyl optionally substituted with —Si(C₁-C₆ alkyl)₃, C₁-C₂₀ haloalkyl, —OH, C₁-C₂₀ alkoxy, C₁-C₂₀ haloalkoxy, hydroxy(C₁-C₂₀ alkyl), alkoxy(C₁-C₂₀ alkyl), —NH₂, —NH(C₁-C₂₀ alkyl), —N(C₁-C₂₀ alkyl)₂, —CONH₂, —CONH(C₁-C₂₀ alkyl), —CON(C₁-C₂₀ alkyl)₂, —NHCO(C₁-C₂₀ alkyl), —NHCO(C₁-C₂₀ alkoxy), —N(C₁-C₂₀ alkyl)CO(C₁-C₂₀ alkyl), —CO₂H, —CO₂(C₁-C₂₀ alkyl), —OCO(C₁-C₂₀ alkyl), —CO₂(aryl), —S(O)₀₋₂—(C₁-C₂₀ alkyl), —S(O)₀₋₂-aryl, —S(O)₀₋₂-heteroaryl, —P(O)(OH)₂, —P(O)(C₁-C₂₀ alkoxy)₂, —P(O)(aryloxy)₂, cycloalkyl, cycloalkyl(C₁-C₂₀ alkyl), aryl, aryl(C₁-C₂₀ alkyl), heteroaryl, heteroaryl(C₁-C₂₀ alkyl), heterocyclyl, and heterocyclyl(C₁-C₂₀ alkyl), or two R⁴ groups on the same non-aromatic atom form an oxo;
R¹ is C₁-C₂₀ alkyl, C₂-C₂₀ alkenyl, C₂-C₂₀ alkynyl optionally substituted with —Si(C₁-C₆ alkyl)₃, C₄-C₁₀ haloalkyl, —CO₂(C₁-C₂₀ alkyl), —S(O)₀₋₂—(C₁-C₂₀ alkyl), —S(O)₀₋₂-aryl, —S(O)₀₋₂-heteroaryl, —P(O)(OH)₂, —P(O)(C₁-C₂₀ alkoxy)₂, —P(O)(aryloxy)₂, aryl, aryl(C₁-C₂₀ alkyl), heteroaryl, or heteroaryl(C₁-C₂₀ alkyl), wherein each of which is independently optionally substituted with one or more R⁵;
R² is C₁-C₂₀ alkyl, C₂-C₂₀ alkenyl, C₂-C₂₀ alkynyl optionally substituted with —Si(C₁-C₆ alkyl)₃, C₄-C₁₀ haloalkyl, —CO₂(C₁-C₂₀ alkyl), —S(O)₀₋₂—(C₁-C₂₀ alkyl), —S(O)₀₋₂-aryl, —S(O)₀₋₂-heteroaryl, —P(O)(OH)₂, —P(O)(C₁-C₂₀ alkoxy)₂, —P(O)(aryloxy)₂, aryl, aryl(C₁-C₂₀ alkyl), heteroaryl, or heteroaryl(C₁-C₂₀ alkyl), wherein each of which is independently optionally substituted with one or more R⁶;
   wherein each R⁵ and R⁶ are independently selected from the group consisting of halogen, —NO₂, —CN, C₁-C₂₀ alkyl, C₂-C₂₀ alkenyl, C₂-C₂₀ alkynyl optionally substituted with —Si(C₁-C₆ alkyl)₃, C₁-C₂₀ haloalkyl, —OH, C₁-C₂₀ alkoxy, C₁-C₂₀ haloalkoxy, hydroxy(C₁-C₂₀ alkyl), alkoxy(C₁-C₂₀ alkyl), —NH₂, —NH(C₁-C₂₀ alkyl), —N(C₁-C₂₀ alkyl)₂, —CONH₂, —CONH(C₁-C₂₀ alkyl), —CON(C₁-C₂₀ alkyl)₂, —NHCO(C₁-C₂₀ alkyl), —N(C₁-C₂₀ alkyl)CO(C₁-C₂₀ alkyl), —CO₂H, —CO₂(C₁-C₂₀ alkyl), —OCO(C₁-C₂₀ alkyl), —CO₂(aryl), —S(O)₀₋₂—(C₁-C₂₀ alkyl), —S(O)₀₋₂-aryl, —S(O)₀₋₂-heteroaryl, —P(O)(OH)₂, —P(O)(C₁-C₂₀ alkoxy)₂, —P(O)(aryloxy)₂, aryl, aryl(C₁-C₂₀ alkyl), heteroaryl, heteroaryl(C₁-C₂₀ alkyl), heterocyclyl, and heterocyclyl(C₁-C₂₀ alkyl), or two R⁵ groups on the same non-aromatic atom form an oxo, or two R⁶ groups on the same non-aromatic atom form an oxo; and
R³ is —CR⁷R⁸R⁹;
   R⁷ is hydrogen or C₁-C₂₀ alkyl optionally substituted with one or more R¹¹;
   R⁸ is hydrogen, C₁-C₂₀ alkyl, aryl, —CO₂R¹⁰, or —CON(R¹⁰)₂; and
   R⁹ is C₄-C₂₀ alkyl, aryl, aryl(C₁-C₂₀ alkyl), heteroaryl, heteroaryl(C₁-C₂₀ alkyl), heterocyclyl, heterocyclyl(C₁-C₂₀ alkyl), —CO₂R¹⁰, —CON(R¹⁰)₂, or —CN, wherein each alkyl, aryl, heteroaryl, or heterocyclyl moiety is optionally substituted with one or more R¹¹; and
   wherein each R¹⁰ is independently selected from the group consisting of hydrogen, C₁-C₂₀ alkyl, or aryl, wherein each alkyl or aryl moiety is optionally substituted with one or more R¹¹;
   wherein each R¹¹ is independently selected from the group consisting of halogen, —NO₂, —CN, C₁-C₂₀ alkyl, C₂-C₂₀ alkenyl, C₂-C₂₀ alkynyl optionally substituted with —Si(C₁-C₆ alkyl)₃, C₁-C₂₀ haloalkyl, —OH, C₁-C₂₀ alkoxy, C₁-C₂₀ haloalkoxy, hydroxy(C₁-C₂₀ alkyl), alkoxy(C₁-C₂₀ alkyl), —NH₂, —NH(C₁-C₂₀ alkyl), —N(C₁-C₂₀ alkyl)₂, —CONH₂, —CONH(C₁-C₂₀ alkyl), —CON(C₁-C₂₀ alkyl)₂, —NHCO(C₁-C₂₀ alkyl), —N(C₁-C₂₀ alkyl)CO(C₁-C₂₀ alkyl), amino(C₁-C₂₀ alkyl), —CO₂H, —CO₂(C₁-C₂₀ alkyl), —OCO(C₁-C₂₀ alkyl), —CO₂(aryl), —S(O)₀₋₂—(C₁-C₂₀ alkyl), —S(O)₀₋₂-aryl, and —S(O)₀₋₂-heteroaryl, or two R¹¹ form an oxo.

The disclosure also provides compounds of formula I or II, wherein
A is selected from aryl and heteroaryl, each of which is independently optionally substituted with one or more R⁴;
   wherein each R⁴ is independently selected from the group consisting of halogen, —NO₂, —CN, C₁-C₂₀ alkyl, C₂-C₂₀ alkenyl, C₂-C₂₀ alkynyl optionally substituted with —Si(C₁-C₆ alkyl)₃, C₁-C₂₀ haloalkyl, —OH, C₁-C₂₀ alkoxy, C₁-C₂₀ haloalkoxy, hydroxy(C₁-C₂₀ alkyl), alkoxy(C₁-C₂₀ alkyl), —NH₂, —NH(C₁-C₂₀ alkyl), —N(C₁-C₂₀ alkyl)₂, —CONH₂, —CONH(C₁-C₂₀ alkyl), —CON(C₁-C₂₀ alkyl)₂, —NHCO(C₁-C₂₀ alkyl), —NHCO(C₁-C₂₀ alkoxy), —N(C₁-C₂₀ alkyl)CO(C₁-C₂₀ alkyl), —CO₂H, —CO₂(C₁-C₂₀ alkyl), —OCO(C₁-C₂₀ alkyl), —CO₂(aryl), —S(O)₀₋₂—(C₁-C₂₀ alkyl), —S(O)₀₋₂-aryl, —S(O)₀₋₂-heteroaryl, —P(O)(OH)₂, —P(O)(C₁-C₂₀ alkoxy)₂, —P(O)(aryloxy)₂, cycloalkyl, cycloalkyl(C₁-C₂₀ alkyl), aryl, aryl(C₁-C₂₀ alkyl), heteroaryl, heteroaryl(C₁-C₂₀ alkyl), heterocyclyl, and heterocyclyl(C₁-C₂₀ alkyl), or two R⁴ groups on the same non-aromatic atom form an oxo;
R¹ is C₁-C₂₀ alkyl, C₂-C₂₀ alkenyl, C₂-C₂₀ alkynyl optionally substituted with —Si(C₁-C₆ alkyl)₃, C₄-C₁₀ haloalkyl, —CO₂(C₁-C₂₀ alkyl), —S(O)₀₋₂—(C₁-C₂₀ alkyl), —S(O)₀₋₂-aryl, —S(O)₀₋₂-heteroaryl, —P(O)(OH)₂, —P(O)(C₁-C₂₀ alkoxy)₂, —P(O)(aryloxy)₂, aryl, aryl(C₁-C₂₀ alkyl), heteroaryl, or heteroaryl(C₁-C₂₀ alkyl), wherein each of which is independently optionally substituted with one or more R⁵;
R² is C₁-C₂₀ alkyl, C₂-C₂₀ alkenyl, C₂-C₂₀ alkynyl optionally substituted with —Si(C₁-C₆ alkyl)₃, C₄-C₁₀ haloalkyl, —CO₂(C₁-C₂₀ alkyl), —S(O)₀₋₂—(C₁-C₂₀ alkyl), —S(O)₀₋₂-aryl, —S(O)₀₋₂-heteroaryl, —P(O)(OH)₂, —P(O)(C₁-C₂₀ alkoxy)₂, —P(O)(aryloxy)₂, aryl, aryl(C₁-C₂₀ alkyl), heteroaryl, or heteroaryl(C₁-C₂₀ alkyl), wherein each of which is independently optionally substituted with one or more R⁶;
   wherein each R⁵ and R⁶ are independently selected from the group consisting of halogen, —NO₂, —CN, C₁-C₂₀ alkyl, C₂-C₂₀ alkenyl, C₂-C₂₀ alkynyl optionally substituted with —Si(C₁-C₆ alkyl)₃, C₁-C₂₀ haloalkyl, —OH, C₁-C₂₀ alkoxy, C₁-C₂₀ haloalkoxy, hydroxy(C₁-C₂₀ alkyl), alkoxy(C₁-C₂₀ alkyl), —NH₂, —NH(C₁-C₂₀ alkyl), —N(C₁-C₂₀ alkyl)₂, —CONH₂, —CONH(C₁-C₂₀ alkyl), —CON(C₁-C₂₀ alkyl)₂, —NHCO(C₁-C₂₀ alkyl), —N(C₁-C₂₀ alkyl)CO(C₁-C₂₀ alkyl), —CO₂H, —CO₂(C₁-C₂₀ alkyl), —OCO(C₁-C₂₀ alkyl), —CO₂(aryl), —S(O)₀₋₂—(C₁-C₂₀ alkyl), —S(O)₀₋₂-aryl, —S(O)₀₋₂-heteroaryl, —P(O)(OH)₂, —P(O)(C₁-C₂₀ alkoxy)₂, —P(O)(aryloxy)₂, cycloalkyl, cycloalkyl(C₁-C₂₀ alkyl), aryl, aryl(C₁-C₂₀ alkyl), heteroaryl, heteroaryl(C₁-C₂₀ alkyl), heterocyclyl, and heterocyclyl(C₁-C₂₀ alkyl), or two R⁵ groups on the same non-aromatic atom form an oxo, or two R⁶ groups on the same non-aromatic atom form an oxo; and
R³ is a polymeric group resulting from polymerization of one or more of vinyl-containing monomers.

Particular embodiments based on formula I or II include those where A is aryl optionally substituted with one or more R⁴. In yet other embodiments based on formula I or II and any one of the preceding embodiments, the disclosure provides for compounds where A is phenyl, naphthyl, or pyrenyl, each optionally substituted with one or more R⁴. In certain embodiments, A is phenyl optionally substituted with one or more R⁴. Other particular embodiments provide for compounds where A is tetrahydronaphthyl, dihydronaphthyl, or dihydroindenyl, each optionally substituted with one or more R⁴.

Particular embodiments based on formula I or II include those where A is heteroaryl optionally substituted with one or more R⁴. In yet other embodiments based on formula I or II and any one of the preceding embodiments, the disclosure provides for compounds where A is pyridinyl, pyrazinyl, pyrimidinyl, pyridazinyl, quinolinyl, isoquinolinyl or benzothiazoyl each optionally substituted with one or more R⁴. In certain embodiments, A is pyridinyl, quinolinyl, isoquinolinyl, or benzothiazoyl, each optionally substituted with one or more R⁴.

Particular embodiments based on formula I or II and any preceding embodiment include those where each R⁴, if present, is selected from the group consisting of halogen, —NO₂, —CN, C₁-C₂₀ alkyl, C₂-C₂₀ alkynyl optionally substituted with —Si(C₁-C₆ alkyl)₃, —OH, C₁-C₂₀ alkoxy, C₁-C₂₀ haloalkoxy, hydroxy(C₁-C₂₀ alkyl), alkoxy(C₁-C₂₀ alkyl), —NH₂, —NH(C₁-C₂₀ alkyl), —N(C₁-C₂₀ alkyl)₂, —CONH₂, —CO₂H, and —CO₂(C₁-C₂₀ alkyl), —CO₂(aryl), —SO₃H, —S(O)₀₋₂—(C₁-C₂₀ alkyl), —P(O)(OH)₂, —P(O)(C₁-C₂₀ alkoxy)₂, —P(O)(aryloxy)₂, aryl, aryl(C₁-C₂₀ alkyl), heteroaryl, or heteroaryl(C₁-C₂₀ alkyl). Other embodiments provide for compounds of formula I or II where each R⁴, if present, is selected from the group consisting of halogen, —CN, C₁-C₂₀ alkyl, C₂-C₂₀ alkynyl optionally substituted with —Si(C₁-C₆ alkyl)₃, —OH, C₁-C₂₀ alkoxy, hydroxy(C₁-C₂₀ alkyl), alkoxy(C₁-C₂₀ alkyl), —CO₂H, and —CO₂(C₁-C₂₀ alkyl), —SO₃H, —P(O)(OH)₂, —P(O)(C₁-C₂₀ alkoxy)₂, aryl, or heteroaryl. In certain embodiments, each R⁴, if present, is selected from the group consisting of halogen, —NO₂, —CN, C₁-C₂₀ alkyl, C₂-C₂₀ alkenyl, C₂-C₂₀ alkynyl optionally substituted with —Si(C₁-C₆ alkyl)₃, C₁-C₂₀ haloalkyl, —OH, C₁-C₂₀ alkoxy, C₁-C₂₀ haloalkoxy, hydroxy(C₁-C₂₀ alkyl), and alkoxy(C₁-C₂₀ alkyl). In other embodiments, each R⁴, if present, is selected from the group consisting of —NO₂, —CN, C₁-C₂₀ alkyl, and C₁-C₂₀ alkoxy. In yet further embodiments, each R⁴, if present, is —CN or C₁-C₂₀ alkyl. For example, R⁴ may be methyl, ethyl, propyl, or butyl.

Embodiments based on formula I or II and any preceding embodiment include those where R¹ is C₁-C₂₀ alkyl, C₂-C₂₀ alkenyl, C₂-C₂₀ alkynyl, aryl, or heteroaryl, wherein each is independently optionally substituted with one or more R⁵. Embodiments based on formula I or II and any preceding embodiment include those where R¹ is C₄-C₂₀ alkyl, C₄-C₂₀ alkenyl, C₄-C₂₀ alkynyl, aryl, or heteroaryl, wherein each is independently optionally substituted with one or more R⁵. Other embodiments provide for compounds of formula I or II where R¹ is C₄-C₁₀ alkyl, aryl, or heteroaryl, wherein each is independently optionally substituted with one or more R⁵. Other embodiments provide for compounds of formula I or II where R¹ is C₂-C₂₀ alkyl, aryl, or heteroaryl, wherein each is independently optionally substituted with one or more R⁵. Certain specific embodiments based on formula I or II and any preceding embodiment include those where R¹ is C₄-C₂₀ alkyl or aryl, wherein each is independently optionally substituted with one or more R⁵. In certain such embodiments, R¹ is aryl optionally substituted with one or more R⁵. In other embodiments, each R⁵, if present, is selected from the group halogen, —NO₂, —CN, C₁-C₂₀ alkyl, C₂-C₂₀ alkenyl, C₂-C₂₀ alkynyl optionally substituted with —Si(C₁-C₆ alkyl)₃, C₁-C₂₀ haloalkyl, —OH, C₁-C₂₀ alkoxy, C₁-C₂₀ haloalkoxy, —NH₂, —NH(C₁-C₂₀ alkyl), and —N(C₁-C₂₀ alkyl)₂. In certain embodiments, each R⁵, if present, is selected from the group consisting of —NO₂, —CN, C₁-C₂₀ alkyl, and C₁-C₂₀ alkoxy. Certain embodiments based on formula I or II and any preceding embodiment include those where R¹ is phenyl, methoxyphenyl, nitrophenyl, cyanophenyl, methylphenyl, trimethylphenyl, triisopropyl, napthyl, or anthracenyl.

Particular embodiments based on formula I or II and any preceding embodiment include those where R² is C₄-C₁₀ alkyl, C₄-C₁₀ alkenyl, C₄-C₁₀ alkynyl, aryl, or heteroaryl, wherein each is independently optionally substituted with one or more R⁶. Other embodiments based on formula I or II and any preceding embodiment include those where R² is C₄-C₂₀ alkyl, C₄-C₂₀ alkenyl, C₄-C₂₀ alkynyl, aryl, or heteroaryl, wherein each is independently optionally substituted with one or more R⁶. Other embodiments provide for compounds of formula I or II where R² is C₁-C₂₀ alkyl, aryl, or heteroaryl, wherein each is independently optionally substituted with one or more R⁶. Some other embodiments provide for compounds of formula I or II where R² is C₄-C₂₀ alkyl, aryl, or heteroaryl, wherein each is independently optionally substituted with one or more R⁶. Certain specific embodiments based on formula I or II and any preceding embodiment include those where R² is C₁-C₂₀ alkyl or aryl, wherein each is independently optionally substituted with one or more R⁶. Other certain specific embodiments based on formula I or II and any preceding embodiment include those where R² is C₄-C₂₀ alkyl or aryl, wherein each is independently optionally substituted with one or more R⁶. In certain such embodiments, R² is aryl optionally substituted with one or more R⁶. In other embodiments, each R⁶, if present, is selected from the group halogen, —NO₂, —CN, C₁-C₂₀ alkyl, C₂-C₂₀ alkenyl, C₂-C₂₀ alkynyl optionally substituted with —Si(C₁-C₆ alkyl)₃, C₁-C₂₀ haloalkyl, —OH, C₁-C₂₀ alkoxy, C₁-C₂₀ haloalkoxy, —NH₂, —NH(C₁-C₂₀ alkyl), and —N(C₁-C₂₀ alkyl)₂. In certain embodiments, each R⁶, if present, is selected from the group consisting of —NO₂, —CN, C₁-C₂₀ alkyl, and C₁-C₂₀ alkoxy. Particular embodiments based on formula I or II and any preceding embodiment include those where R² is unsubstituted phenyl. Certain embodiments based on formula I or II and any preceding embodiment include those where R² is phenyl, methoxyphenyl, nitrophenyl, cyanophenyl, methylphenyl, trimethylphenyl, triisopropyl, napthyl, or anthracenyl.

Embodiments based on formula I or II and any preceding embodiment include those where R³ is —CR⁷R⁸R⁹; and R⁷ is hydrogen. Other embodiments based on formula I or II and any preceding embodiment provide for compounds where R⁷ is C₁-C₂₀ alkyl optionally substituted with one or more R¹¹. In other certain embodiments, R⁷ is methyl.

Embodiments based on formula I or II and any preceding embodiment include those where R³ is —CR⁷R⁸R⁹; and R⁸ is hydrogen, C₁-C₂₀ alkyl, —CO₂R¹⁰, or —CON(R¹⁰)₂, wherein alkyl is optionally substituted with one or more R¹¹. Certain embodiments provide for compounds where R⁸ is hydrogen. Other embodiments provide for compounds where R⁸ is C₁-C₂₀ alkyl. In particular embodiments, R⁸ is methyl. In certain embodiments, R⁸ is —CO₂R¹⁰ or —CON(R¹⁰)₂. Other embodiments provide for compounds where R⁸ is —CO₂H, —CO₂(C₁-C₂₀ alkyl), —CONH(C₁-C₂₀ alkyl), or —CON(C₁-C₂₀ alkyl)₂.

Particular embodiments based on formula I or II and any preceding embodiment include those where R³ is —CR⁷R⁸R⁹; R⁹ is C₄-C₂₀ alkyl, aryl, heteroaryl, —CO₂R¹⁰, or —CON(R¹⁰)₂, wherein each alkyl, aryl, or heteroaryl moiety is optionally substituted with one or more R¹¹. Other particular embodiments based on formula I or II and any preceding embodiment include those where R⁹ is C₄-C₂₀ alkyl, aryl, heteroaryl, —CO₂R¹⁰, or —CON(R¹⁰)₂, wherein each alkyl, aryl, or heteroaryl moiety is optionally substituted with one or more R¹¹. Certain embodiments provide for compounds where R⁹ is C₄-C₂₀ alkyl. Certain embodiments provide for compounds where R⁹ is aryl, —CO₂R¹⁰, or —CON(R¹⁰)₂, wherein each moiety is optionally substituted with one or more R¹¹. Other embodiments provide for compounds where R⁹ is aryl optionally substituted with one or more R¹¹. In particular embodiments, R⁹ is phenyl. In certain embodiments, R⁹ is —CO₂R¹⁰ or —CON(R¹⁰)₂. Other embodiments provide for compounds where R⁹ is —CO₂H, —CO₂(C₁-C₂₀ alkyl), —CONH(C₁-C₂₀ alkyl), or —CON(C₁-C₂₀ alkyl)₂.

Certain specific embodiments based on formula I or II include those where R³ is —CR⁷R⁸R⁹; where R⁷ is hydrogen or C₁-C₂₀ alkyl; R⁸ is C₁-C₂₀ alkyl; and R⁹ is aryl optionally substituted with one or more R¹¹.

Certain specific embodiments based on formula I or II include those where R³ is —CR⁷R⁸R⁹; wherein R⁷ is hydrogen or C₁-C₂₀ alkyl; R⁸ is C₁-C₂₀ alkyl, —CO₂R¹⁰, or —CON(R¹⁰)₂; and R⁹ is —CO₂R¹⁰, or —CON(R¹⁰)₂,

Particular embodiments based on formula I or II and any preceding embodiment include those where R³ is selected from the group consisting of:

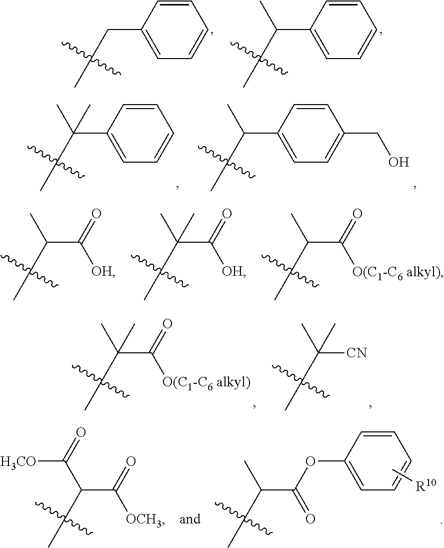

Certain non-limiting exemplary embodiments provide compounds of formula I or II, wherein
A is selected from aryl or heteroaryl, each of which is independently optionally substituted with one or more R⁴;
R¹ is C₄-C₁₀ alkyl, C₄-C₁₀ alkenyl, C₄-C₁₀ alkynyl, C₄-C₁₀ haloalkyl, —CO₂(C₁-C₂₀ alkyl), —S(O)₀₋₂—(C₁-C₂₀ alkyl), —S(O)₀₋₂-aryl, —S(O)₀₋₂-heteroaryl, aryl, aryl(C₁-C₂₀ alkyl), heteroaryl, or heteroaryl(C₁-C₂₀ alkyl), wherein each of which is independently optionally substituted with one or more R⁵;
R² is C₄-C₁₀ alkyl, C₄-C₁₀ alkenyl, C₄-C₁₀ alkynyl, C₄-C₁₀ haloalkyl, —CO₂(C₁-C₂₀ alkyl), —S(O)₀₋₂—(C₁-C₂₀ alkyl), —S(O)₀₋₂-aryl, —S(O)₀₋₂-heteroaryl, aryl, aryl(C₁-C₂₀ alkyl), heteroaryl, or heteroaryl(C₁-C₂₀ alkyl), wherein each of which is independently optionally substituted with one or more R⁶; and
R³ is —CR⁷R⁸R⁹;
   R⁷ is hydrogen or C₁-C₂₀ alkyl optionally substituted with one or more R¹¹;
   R⁸ is hydrogen, C₁-C₂₀ alkyl, aryl, —CO₂R¹⁰, or —CON(R¹⁰)₂; and
   R⁹ is C₄-C₂₀ alkyl, aryl, aryl(C₁-C₂₀ alkyl), heteroaryl, heteroaryl(C₁-C₂₀ alkyl), —CO₂R¹⁰ or —CON(R¹⁰)₂, wherein each alkyl, aryl, heteroaryl, or heterocyclyl moiety is optionally substituted with one or more R¹¹; and
   wherein each R¹⁰ is independently selected from the group consisting of hydrogen, C₁-C₂₀ alkyl, or aryl, wherein each alkyl or aryl moiety is optionally substituted with one or more R¹¹;
   wherein each R¹¹ is independently selected from the group consisting of halogen, —NO₂, —CN, C₁-C₂₀ alkyl, C₁-C₂₀ haloalkyl, —OH, C₁-C₂₀ alkoxy, C₁-C₂₀ haloalkoxy, hydroxy(C₁-C₂₀ alkyl), alkoxy(C₁-C₂₀ alkyl), amino(C₁-C₂₀ alkyl), —CO₂H, —CO₂(C₁-C₂₀ alkyl), —OCO(C₁-C₂₀ alkyl), —CO₂(aryl), —S(O)₀₋₂—(C₁-C₂₀ alkyl), —S(O)₀₋₂-aryl, and —S(O)₀₋₂-heteroaryl, or two R¹¹ form an oxo.

Certain non-limiting exemplary embodiments provide compounds of formula I or II, wherein
A is aryl or heteroaryl, each independently optionally substituted with one or more R⁴;
R¹ is C₄-C₁₀ alkyl, aryl, or heteroaryl, wherein each is independently optionally substituted with one or more R⁵;
R² is C₄-C₁₀ alkyl, aryl, or heteroaryl, wherein each is independently optionally substituted with one or more R⁶; and
R³ is —CR⁷R⁸R⁹; wherein
   R⁷ is hydrogen or C₁-C₂₀ alkyl optionally substituted with one or more R¹¹;
   R⁸ is C₁-C₂₀ alkyl, —CO₂R¹⁰, or —CON(R¹⁰)₂, wherein each is optionally substituted with one or more R¹¹; and
   R⁹ is aryl, heteroaryl, —CO₂R¹⁰, or —CON(R¹⁰)₂, wherein each is optionally substituted with one or more R¹¹.

Other non-limiting exemplary embodiments provide compounds of formula I or II, wherein

A is aryl optionally substituted with one or more R⁴;
R¹ is C₄-C₁₀ alkyl or aryl, each independently optionally substituted with one or more R⁵;
R² is C₄-C₁₀ alkyl or aryl, each independently optionally substituted with one or more R⁶; and
R³ is —CR⁷R⁸R⁹;
R⁷ is hydrogen or C₁-C₂₀ alkyl optionally substituted with one or more R¹¹;
R⁸ is C₁-C₂₀ alkyl, —CO₂R¹⁰, or —CON(R¹⁰)₂, wherein each is optionally substituted with one or more R¹¹; and
R⁹ is aryl, —CO₂R¹⁰, or —CON(R¹⁰)₂, wherein each is optionally substituted with one or more R¹¹.

Other non-limiting exemplary embodiments provide compounds of formula I or II, wherein

A is aryl optionally substituted with one or more R⁴;
R¹ is C₄-C₁₀ alkyl or aryl, each independently optionally substituted with one or more R⁵;
R² is C₄-C₁₀ alkyl or aryl, each independently optionally substituted with one or more R⁶; and
R³ is —CR⁷R⁸R⁹; wherein
R⁷ is C₁-C₂₀ alkyl optionally substituted with one or more R¹¹;
R⁸ is C₁-C₂₀ alkyl, —CO₂R¹⁰, or —CON(R¹⁰)₂, wherein each alkyl is optionally substituted with one or more R¹¹; and
R⁹ is aryl optionally substituted with one or more R¹¹.

Other non-limiting exemplary embodiments provide compounds of formula I or II, wherein
A is aryl optionally substituted with one or more R⁴;
R¹ is C₄-C₁₀ alkyl or aryl, each independently optionally substituted with one or more R⁵;
R² is C₄-C₁₀ alkyl or aryl, each independently optionally substituted with one or more R⁶;
R⁷ is C₁-C₂₀ alkyl optionally substituted with one or more R¹¹;
R⁸ is C₁-C₂₀ alkyl, —CO₂R¹⁰, or —CON(R¹⁰)₂, wherein each alkyl optionally substituted with one or more R¹¹; and
R⁹ is —CO₂R¹⁰ or —CON(R¹⁰)₂.

Particular embodiments based on formula I or II and any preceding embodiment include those where each C₁-C₂₀ alkyl moiety (including alkyl moieties on any group, such as, for example, amine, alkoxy, or sulfonyl groups) is independently C₁-C₁₂ alkyl; or C₁-C₁₀ alkyl; or C₁-C₈ alkyl; or C₁-C₆ alkyl. Other particular embodiments based on formula I or II and any preceding embodiment include those where each C₂-C₂₀ alkenyl is independently C₂-C₁₂ alkenyl; or C₂-C₁₀ alkenyl; or C₂-C₈ alkenyl; or C₂-C₆ alkenyl. Other particular embodiments based on formula I or II and any preceding embodiment include those where each C₂-C₂₀ alkynyl is independently C₂-C₁₂ alkynyl; or C₂-C₁₀ alkynyl; or C₂-C₈ alkynyl; or C₂-C₆ alkynyl.

Particular embodiments based on formula I or II and any preceding embodiment include those where R³ is a polymeric group resulting from polymerization of one or more of vinyl-containing monomers. In certain embodiments, R³ is a polymer resulting from polymerization of one or more of optionally substituted styrenes, optionally substituted alkylacrylates, optionally substituted alkylmethacrylates, acrylonitrile, methacrylonitrile, acrylamide, methacrylamide, isoprene, butadiene, ethylene, vinylacetate, vinyl ethers, and their combinations. In other embodiments, R³ is a polymer resulting from polymerization of one or more of vinyl-containing monomers specifically disclosed below in Table A. These polymeric groups may have molecular weights of from about 200 to about 100,000 Da; or about 200 to about 50,000 Da; or from about 500 to about 50,000 Da; or from about 500 to about 30,000 Da; or from about 1,000 to about 20,000 Da; or from about 500 to about 10,000 Da.

Non-limiting examples of this embodiment include compounds of formula:

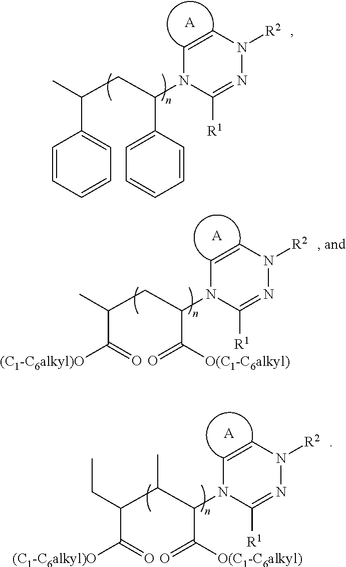

Another aspect of the disclosure provides for methods for polymerizing one or more vinyl-containing monomers comprising contacting one or more vinyl-containing monomers with one or more compounds of the disclosure.

The compounds of the disclosure are used as polymerization mediators in the methods of the invention. One or more of the compounds of the disclosure may be used in the methods of the disclosure. In certain embodiments, the reaction mixtures employed in the methods of the invention are free or substantially free of any additional (secondary) polymerization mediator. In certain embodiments, the reaction mixtures employed in the methods of the invention are free or substantially free of an initiator. By “substantially free” as used herein is meant containing less than 0.1, or less than 0.05, or less than 0.01, or less than 0.001 molar equivalents.

In one embodiment, the methods of the disclosure provide for radical-mediated polymerization. In another embodiment, the methods of the disclosure provide for controlled radical-mediated polymerization. The degree of polymerization is the number average molecular weight divided by the weighted average molecular weight of all monomers in the feed, which; in a controlled polymerization, the number average molecular weight is a linear function of monomer conversion. Controlled radical polymerization requires: sufficiently fast initiation so that nearly all chains start to grow simultaneously; and little or no chain transfer. A broad polydispersity index (PDI) of a polymer indicates that the polymer contains polymeric segments with substantial smaller and larger molecular weight segments than the number average molecular weight of the polymer. Low molecular weight segments may have an adverse effect on physical properties of the polymer such as tensile strength, elongation and flexural modulus; while very large molecular weight segments may result in high melt viscosity of the polymer which may produce limitations in the processability of the polymer.

Thus, there are advantages for the final polymer to have a well-defined and narrow PDI. As used herein, the term “controlled radical polymerization” or “controlled radical-mediated polymerization” is polymerization where the resulting polymer has PDI of less than about 1.5. In some embodiments, the PDI of the resulting polymer is less than about 1.3.

The methods of the invention allow for greater control over the final polymer products such that the desired chain length, polydispersity, molecular weight, and functionality are easily incorporated into the final product. Thus, the present invention overcomes the poor control over molecular weight distribution, low functionality, poor control of polymer rheology, and undesirable polydispersity. The methods of the disclosure may also be implemented on a large scale with a high predictability and/or used to tailor the properties of the final polymer products to new degrees, and products can be designed based on their properties.

Suitable vinyl-containing monomers used in the methods and compositions of the disclosure are any ethylene-containing monomers, and can be chosen from the group consisting of styrene, substituted styrenes, substituted or unsubstituted alkyl(meth)acrylates, acrylonitrile, methacrylonitrile, acrylamide, methacrylamide, derivatives mono- and di-substituted on the nitrogen of the acrylamide and of the methacrylamide, isoprene, butadiene, ethylene, vinylacetate, vinyl ethers, and their combinations.

The specific monomers and co-monomers which can be used in the invention include methyl methacrylate, ethyl methacrylate, propyl methacrylate (all the isomers), butyl methacrylate (all the isomers), 2-ethylhexyl methacrylate, isobornyl methacrylate, methacrylic acid, benzyl methacrylate, phenyl methacrylate, methacrylo-nitrile, .alpha.-methylstyrene, methyl acrylate, ethyl acrylate, propyl acrylate (all the isomers), butyl acrylate (all the isomers), 2-ethylhexyl acrylate, isobornyl acrylate, acrylic acid, benzyl acrylate, phenyl acrylate, acrylonitrile, styrene, glycidyl methacrylate, 2-hydroxyethyl methacrylate, hydroxy-propyl methacrylate (all the isomers), hydroxybutyl methacrylate (all the monomers), N,N-dimethylaminoethyl methacrylate, N,N-diethylaminoethyl methacrylate, triethylene glycol methacrylate, N-methacryloyloxy-succinimide, itaconic anhydride, itaconic acid, glycidyl acrylate, 2-hydroxyethyl acrylate, hydroxy-propyl acrylate (all the isomers), hydroxybutyl acrylate (all the isomers), N,N-dimethylaminoethyl acrylate, N,N-diethylaminoethyl acrylate, triethylene glycol acrylate, N-acryloyloxysuccinimide, methacrylamide, N-methylacrylamide, N, N-dimethylacrylamide, N-(tert-butyl)methacrylamide, N-(n-butyl)methacrylamide, N-methylolmethacrylamide, N-ethylolmethacrylamide, N-(tert-butyl)acrylamide, N-octadecylacrylamide, N-methylolacrylamide, N-ethylolacrylamide, N-acryloyl-morpholine, vinylbenzoic acid (all the isomers), diethylaminostyrene (all the isomers), α-methylvinyl-benzoic acid (all the isomers), diethylamino-α-methylstyrene (all the isomers), the acid or the sodium salt of p-vinylbenzenesulfonic acid, trimethoxysilylpropyl methacrylate, tributoxysilylpropyl methacrylate, the dimethoxymethylsilylpropyl methacrylate, diethoxymethylsilylpropyl methacrylate, dibutoxymethylsilylpropyl methacrylate, diisopropoxymethylsilylpropyl methacrylate, dimethoxysilylpropyl methacrylate, diethoxysilylpropyl methacrylate, dibutoxysilylpropyl methacrylate, diisopropoxysilylpropyl methacrylate, trimethoxysilylpropyl acrylate, tributoxysilylpropyl acrylate, dimethoxymethylsilylpropyl acrylate, diethoxymethylsilylpropyl acrylate, dibutoxymethylsilylpropyl acrylate, diisopropoxymethylsilylpropyl acrylate, dimethoxysilylpropyl acrylate, diethoxysilylpropyl acrylate, dibutoxysilylpropyl acrylate, diisopropoxysilylpropyl acrylate, maleic anhydride, N-phenylmaleimide, N-butylmaleimide, N-vinylpyrrolidone, butadiene, isoprene, chloroprene, ethylene, vinyl acetate and their combinations.

In specific embodiments, the vinyl-containing monomers that may be used in the methods of the invention include one or more of those shown below in Table A:

TABLE A

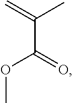

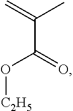

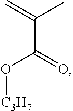

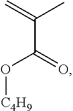

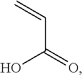

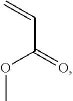

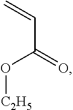

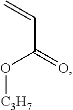

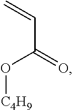

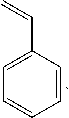

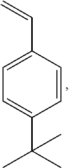

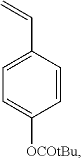

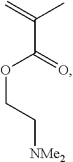

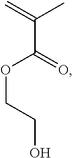

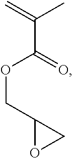

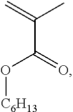

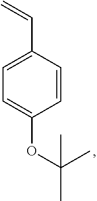

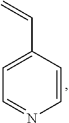

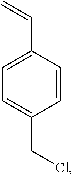

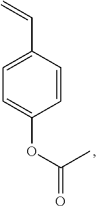

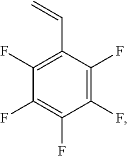

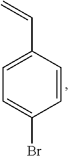

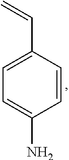

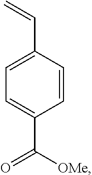

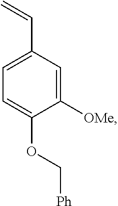 [SMILES: C=Cc1ccc(OCc2ccccc2)c(OC)c1]

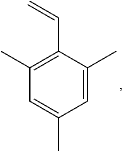

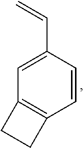

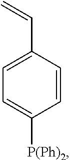

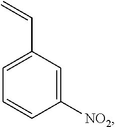

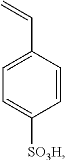

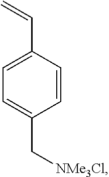

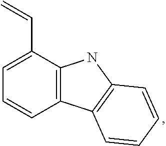

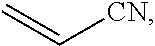

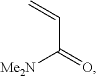

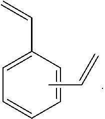

Combinations of the vinyl-containing monomers shown above in Table A may also be used in the invention. In certain embodiments, a mixture of different vinyl-containing monomers can be employed in the invention. In another embodiment, the reaction mixtures comprises one vinyl-containing monomer.

In various embodiments, the methods disclosed herein are conducted at a temperature within the range of about 30° C. to about 300° C., or of about 80° C. to about 250° C., or of about 100° C. to about 200° C., or of about 110° C. to about 150° C., or of about 120° C. to about 140° C., or of about 120° C. to about 130° C., or of about 120° C., or about 125° C., or about 130° C. The reaction may last, for example, for a time within the range of about 1 to about 48 hours, or about 1 to about 24 hours, or about 2 to about 12 hours, or about 2 to about 7 hours, or about 3 to about 5 hours, e.g., about 3 hours, about 4 hours, about 5 hours, about 6 hours, or about 7 hours.

In various embodiments of the methods disclosed herein, the polymerization may be performed in bulk, solution, emulsion, miniemulsion, or suspension. In particular embodiments, methods of the disclosure are performed in bulk. In other particular embodiments, methods of the disclosure are performed in solution.

Solvents suitable for use in the methods disclosed herein include, but are not limited to, water, methanol, ethanol, propanol, isopropanol, butanol, tert butanol, amyl alcohol, tert-amyl alcohol, octanol, furfurol, ethanolamines, glycerine, natural or synthetic polymeric alcohols, ethylene glycol, diethylene glycol, triethylene glycol, 2-(2-ethoxyethoxy)ethanol, tetraethylene glycol, HMPA, phenols, DMSO, DMF, DMAc, NMP, 1-ethyl-2-pyrrolidone, N-methyl-2-piperidone, N-methylcaprolactam, dipolar aprotic solvents, ethylene carbonate, propylene carbonate, ionic liquid, pentane, isooctane, cyclohexane, hexane, heptane, decane, decalin, petroleum ether, benzene, toluene, xylene, mesitylene, ethylbenzene, tetrahydrofuran, 2-methyl tetrahydrofuran, diethylether, diisopropylether, methyl tert-butyl ether, cyclopropyl methyl ether, dimethoxyethane, diethoxyethane, dibutylethane, gamma-butyrolactone, acetone, pentanone, dioxane, chloroform, dichloromethane, carbon tetrachloride, dichloroethane, 1,2-dichlorobenzene, anisol, 1,2-methyl benzene, trifluoromethyltoluene, ethyl acetate, tert-butyl acetate, acetonitrile, benzonitrile, butylnitrile, tert-butylnitrile, isopropylnitrile, propylnitrile, triethylamine, pyridine, acetic acid, trifluoroacetic acid, or a mixture thereof.

In certain embodiments, the reaction mixtures employed in the methods of the invention further comprise one or more additives. Suitable additives include, but are not limited to, organic acids (such as camphorsulfonic acid, 2-fluoro-1-methylpyridinium-p-toluene sulfonate, sulfuric acid), reducing agents (such as ascorbic acid, ascorbic-6-palmitate, benzoin, anisoin, hydroxyacetone), reducing sugars (such as glucose, glyceraldehyde, galactose, lactose, maltose, and fructose). In certain embodiments, the additive may comprise α-hydroxy ketones and aldehydes (such as 3-hydroxy-2-butanone, alpha-hydroxy-gamma-butyrolactone, glycolaldehyde dimer, or glyceraldehyde dimer) that can produce reducing species in the presence of organic bases (pyridine, imidazole, and DMAP), such as glyceraldehyde dimer in combination with pyridine. In other certain embodiments, the additive may be one or more of radical initiators, such as t-butyl hydroperoxide, dicumyl peroxide, azobisisobutyronitrile (AIBN) and other diazoinitiators.

### DEFINITIONS

The following terms and expressions used have the indicated meanings.

Terms used herein may be preceded and/or followed by a single dash, “-”, or a double dash, “=”, to indicate the bond order of the bond between the named substituent and its parent moiety; a single dash indicates a single bond and a double dash indicates a double bond. “ [symbol] ” means a single or double bond. In the absence of a single or double dash it is understood that a single bond is formed between the substituent and its parent moiety; further, substituents are intended to be read “left to right” unless a dash indicates otherwise. For example, C₁-C₆alkoxycarbonyloxy and —OC(O)C₁-C₆alkyl indicate the same functionality; similarly arylalkyl and -alkylaryl indicate the same functionality.

The term “alkenyl” as used herein, means a straight or branched chain hydrocarbon containing from 2 to 20 carbons, unless otherwise specified, and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and 3,7-dimethylocta-2,6-dienyl, and 2-propyl-2-heptenyl. The term “alkenylene” refers to a divalent alkenyl group, where alkenyl is as defined herein.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkyl” as used herein, means a straight or branched chain hydrocarbon containing from 1 to 20 carbon atoms unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. The term “alkylene” refers to a divalent alkyl group, where alkyl is as defined herein.

The term “alkynyl” as used herein, means a straight or branched chain hydrocarbon group containing from 2 to 10 carbon atoms unless otherwise specified, and containing at least one carbon-carbon triple bond. Representative examples of alkynyl include, but are not limited, to acetylenyl, 1-propynyl, 2-propynyl, 3-butynyl, 2-pentynyl, and 1-butynyl. The term “alkynylene” refers to a divalent alkynyl group, where alkynyl is as defined herein.

The term “aryl,” as used herein, means a phenyl (i.e., monocyclic aryl), or a bicyclic ring system containing at least one phenyl ring or an aromatic bicyclic ring containing only carbon atoms in the aromatic bicyclic ring system, or a polycyclic ring system containing at least one phenyl ring. The bicyclic aryl can be azulenyl, naphthyl, or a phenyl fused to a cycloalkyl, a cycloalkenyl, or a heterocyclyl. The bicyclic or polycyclic aryl is attached to the parent molecular moiety through any carbon atom contained within the phenyl portion of the bicyclic or polycyclic system, or any carbon atom with the napthyl, azulenyl, anthracene, or pyrene ring.

The terms “cyano” and “nitrile” as used herein, mean a —CN group.

The term “cycloalkyl” as used herein, means a monocyclic or a bicyclic cycloalkyl ring system. Monocyclic ring systems are cyclic hydrocarbon groups containing from 3 to 10 carbon atoms, where such groups can be saturated or unsaturated, but not aromatic. In certain embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane.

The term “halogen” as used herein, means —Cl, —Br, —I or —F.

The terms “haloalkyl”, “haloalkenyl” and “haloalkoxy” refer to an alkyl, alkenyl or alkoxy group, as the case may be, which is substituted with one or more halogen atoms.

The term “heteroaryl,” as used herein, means a monocyclic heteroaryl or a bicyclic or polycyclic ring system containing at least one heteroaromatic ring. The monocyclic heteroaryl can be a 5 or 6 membered ring. The 5 membered ring consists of two double bonds and one, two, three or four nitrogen atoms and optionally one oxygen or sulfur atom. The 6 membered ring consists of three double bonds and one, two, three or four nitrogen atoms. The 5 or 6 membered heteroaryl is connected to the parent molecular moiety through any carbon atom or any nitrogen atom contained within the heteroaryl. The bicyclic or polycyclic heteroaryl consists of a heteroaryl fused to a phenyl, a cycloalkyl, a cycloalkenyl, a heterocyclyl, or a heteroaryl. Representative examples of heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, triazinyl, benzimidazolyl, benzofuranyl, benzothienyl, benzoxadiazolyl, benzoxathiadiazolyl, benzothiazolyl, cinnolinyl, 5,6-dihydroquinolin-2-yl, 5,6-dihydroisoquinolin-1-yl, furopyridinyl, indazolyl, indolyl, isoquinolinyl, naphthyridinyl, quinolinyl, or purinyl.

The term “heterocyclyl” as used herein, means a monocyclic heterocycle or a bicyclic heterocycle. The monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The bicyclic heterocycle is a monocyclic heterocycle fused to either a phenyl, a monocyclic cycloalkyl, a monocyclic cycloalkenyl, a monocyclic heterocycle, or a monocyclic heteroaryl. Representative examples of heterocycle include, but are not limited to, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, maleimidyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, trithianyl, 2,3-dihydrobenzofuran-2-yl, and indolinyl.

The phrase “one or more” substituents, as used herein, refers to a number of substituents that equals from one to the maximum number of substituents possible based on the number of available bonding sites, provided that the above conditions of stability and chemical feasibility are met. Unless otherwise indicated, an optionally substituted group may have a substituent at each substitutable position of the group, and the substituents may be either the same or different. As used herein, the term “independently selected” means that the same or different values may be selected for multiple instances of a given variable in a single compound.

“Optional” or “optionally” means that the subsequently described event or circumstance may or may not occur, and that the description includes instances where said event or circumstance occurs and instances in which it does not. One of ordinary skill in the art would understand that with respect to any molecule described as containing one or more optional substituents, only sterically practical and/or synthetically feasible compounds are meant to be included. “Optionally substituted” refers to all subsequent modifiers in a term, unless stated otherwise.

The term “polymer” as used herein, is synonymous with “copolymer”, “heteropolymer” and “alternating copolymer” and means a large molecule (macromolecule) composed of a repeating series of one or more alternating monomeric species. These sub-units are typically connected by covalent chemical bonds.

The term “substituted”, as used herein, means that a hydrogen radical of the designated moiety is replaced with the radical of a specified substituent, provided that the substitution results in a stable or chemically feasible compound. The term “substitutable”, when used in reference to a designated atom, means that attached to the atom is a hydrogen radical, which can be replaced with the radical of a suitable substituent.

### EXAMPLES

The preparation of the compounds of the disclosure is illustrated further by the following examples, which are not to be construed as limiting the disclosure in scope or spirit to the specific procedures and compounds described in them. In all cases, unless otherwise specified, the column chromatography is performed using a silica gel solid phase.

Those having skill in the art will recognize that the starting materials and reaction conditions may be varied, the sequence of the reactions altered, and additional steps employed to produce compounds encompassed by the present disclosure, as demonstrated by the following examples. Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Starting materials can be obtained from commercial sources or prepared by well-established literature methods known to those of ordinary skill in the art. The reactions are performed in a solvent appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the disclosure.

In some cases, protection of certain reactive functionalities may be necessary to achieve some of the above transformations. In general, the need for such protecting groups as well as the conditions necessary to attach and remove such groups will be apparent to those skilled in the art of organic synthesis. An authoritative account describing the many alternatives to the trained practitioner are J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie”, Houben-Weyl, 4.sup.th edition, Vol. 15/I, Georg Thieme Verlag, Stuttgart 1974, in H.-D. Jakubke and H. Jescheit, “Aminosauren, Peptide, Proteine”, Verlag Chemie, Weinheim, Deerfield Beach, and Basel 1982, and/or in Jochen Lehmann, “Chemie der Kohlenhydrate: Monosaccharide and Derivate”, Georg Thieme Verlag, Stuttgart 1974. The protecting groups may be removed at a convenient subsequent stage using methods known from the art.

The disclosures of all articles and references mentioned in this application, including patents, are incorporated herein by reference in their entirety.

### Material and Equipment

All reactions were run under Argon unless noted. Benzoyl peroxide (BPO, Aldrich, 97%) was used as received. N, N, N′, N′, N″-Pentamethyldiethylenetriamine (Aldrich, 99%), Cu(0) (Aldrich, 99%), CuBr (Aldrich, 99.999%). Monomers were passed through a column of basic alumina to remove inhibitors before use.

Nuclear magnetic resonance spectra were recorded on a Varian 400 MHz, a Varian 500 MHz or a Varian 600 MHz instrument. All ¹H NMR experiments are reported in δ units, parts per million (ppm), and were measured relative to the signals for residual chloroform (7.26 ppm) in the deuterated solvent, unless otherwise stated. All ¹³C NMR spectra are reported in ppm relative to deuterochloroform (77.23 ppm), unless otherwise stated, and all were obtained with ¹H decoupling. VG70 Magnetic Sector and Waters GCT Premier TOF instruments were used for low and high resolution mass analysis by electron ionization (EI). Micromass QTOF2 Quadrupole/Time-of-Flight Tandem mass spectrometer was used for high-resolution mass analysis using electrospray ionization (ESI). Gel permeation chromatography (GPC) was performed on a Waters 2695 separation module with a Waters 2414 refractive index detector in chloroform with 0.25% triethylamine. Number average molecular weights (Mₙ) and weight average molecular weights (M_{w}) were calculated relative to linear polystyrene standards.

### Examples 1-10

The compounds of Examples 1-10 are prepared according to the scheme outlined below.

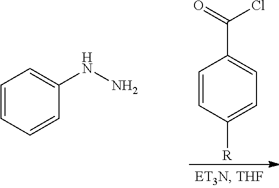

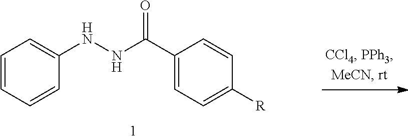

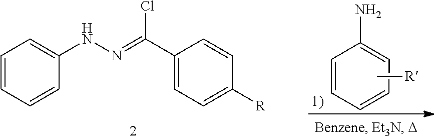

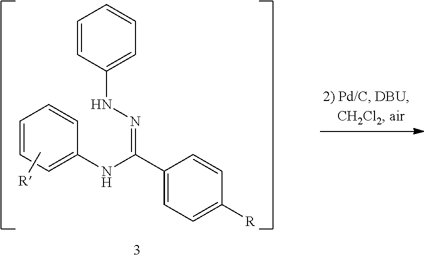

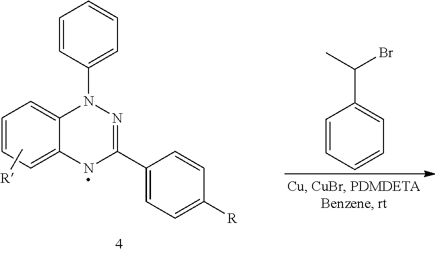

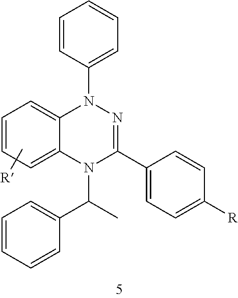

| Ex. | Entry | R | R′ |
|---|---|---|---|
| 1 | 5a | H | H |
| 2 | 5b | OMe | H |
| 3 | 5c | CN | H |
| 4 | 5d | H | CN |
| 5 | 5e | NO₂ | H |
| 6 | 5f | H | —N═CH—CH═CH— |
| 7 | 5g | H | —CH═CH—CH═CH— |
| 8 | 5h | H | CH₃ |
| 9 | 5i | H | CH₂CH₃ |
| 10 | 5j | H | C(CH₃)₃ |

### General Procedure A for the Preparation of Benzoyl Hydrazine 1 (a-c and e)

Triethylamine (12.8 mL, 92.5 mmol) was added to a solution of phenylhydrazine (5 g, 46.3 mmol) in THF (60 mL) at 0° C. The resulting mixture was stirred at 0° C. for 10 min and benzoyl chloride (46.3 mmol) in THF (30 mL) was added dropwise. The reaction mixture was then stirred for 18 h and was slowly warmed to room temperature. Then the solvent was evaporated under reduced pressure and the residue was dissolved in ethyl acetate (150 ml), washed with water (2×100 ml) and dried over MgSO₄. The solvent was removed in vacuo. Recrystallization from minimum amount of dichloromethane gave rise to 1(a-j).

N-phenylbenzoylhydrazide 1a: Colorless solid, yield 60%, ¹H NMR (500 MHz, DMSO-d₆), δ 10.35 (d, J=2.7 Hz, NH, 1H), 7.90 (m, 2H), 7.88 (d, J=2.6 Hz, NH, 1H), 7.56 (m, 1H), 7.49 (m, 2H), 7.14 (dd, J=8.5, 7.2 Hz, 2H), 6.78 (m, 2H), 6.70 (m, 1H), ¹³C NMR (126 MHz, DMSO-d₆) δ 166.79, 149.95, 133.46, 132.08, 129.19, 128.93, 127.73, 119.09, 112.77, HR-ESI C₁₃H₁₂N₂O (M+Na)+ cal. 235.0847. found 235.0832. IR (neat) 3268, 3056, 1642, 1600, 1494, 1481, 1303, 1205, 901, 750

N′-(4-methoxyphenyl)benzohydrazide 1b: Colorless solid, yield 50%, ¹H NMR (500 MHz, CDCl₃) δ 7.91 (br, NH, 1H), 7.81 (d, J=8.8 Hz, 2H), 7.24 (m, 2H), 6.96-6.91 (m, 5H), 6.34 (br, NH, 1H), 3.86 (s, 3H), ¹³C NMR (151 MHz, CDCl₃) δ 162.74, 148.16, 129.17, 128.97, 124.44, 121.31, 113.98, 113.76, 55.44. HR-ESI C₁₄H₁₄N₂O₂ cal. 242.1055. found 242.1065. IR (neat) 3261, 1636, 1601, 1494, 1247, 1172, 1027, 903, 843, 751

N′-(4-cyanophenyl)benzohydrazide 1c: Yellow solid, yield 51%, 1H NMR (500 MHz, DMSO-d₆) δ 10.59 (d, J=2.7 Hz, NH, 1H), 8.05 (d, J=8.4 Hz, 2H), 7.98 (d, J=8.3 Hz, 3H), 7.14 (dd, J=8.5, 7.2 Hz, 2H), 6.78 (d, J=7.3 Hz, 2H), 6.72 (dd, J=7.3, 1.1 Hz, 1H), ¹³C NMR (126 MHz, DMSO-d₆) δ 165.46, 149.57, 137.48, 133.05, 129.24, 128.62, 119.31, 118.73, 114.47, 112.82. HR-ESI C₁₄H₁₁N₃O (M+Na)+ cal. 260.0794. found 260.0791. IR (neat) 3243, 2232, 1648, 1600, 1493, 1307, 1250, 904, 862, 747

N′-(4-nitrophenyl)benzohydrazide) 1e: Orange solid, 67%, 1H NMR (500 MHz, DMSO-d₆) δ 10.67 (s, NH, 1H), 8.34 (d, J=8.8 Hz, 2H), 8.14 (d, J=8.9 Hz, 2H), 7.15 (dd, J=8.5 Hz, 2H), 6.86 (d, J=7.5 Hz, 2H), 6.75 (m, 1H), ¹³C NMR (126 MHz, DMSO-d₆) δ 165.2, 149.7, 149.5, 139.1, 129.3, 129.2, 124.1, 119.1, 112.8, HR-ESI C₁₃H₁₁N₃O (M+Na)+ cal. 280.0698. found 280.0693.

General Procedure B for the Preparation of Benzohydrazonoyl Chloride 2(a-c and e)

Under a flow of nitrogen, to a suspension of compound 2a-c (22.0 mmol) in anhydrous acetonitrile (60 mL) were added triphenylphosphine (27.2 mmol) and anhydrous carbon tetrachloride (27.2 mmol) and left to react overnight at room temperature. Afterwards solvent was evaporated under reduced pressure and the crude product was purified by chromatography on silica gel.

(E/Z)—N′-phenylbenzohydrazonoyl chloride 2a: Compound 2a was obtained according to procedure disclosed in Zhang, C. Y. et al., Chem. Biol. Drug. Des. 2010, 75, 489-493.

(E/Z)—N′-(4-methoxyphenyl)benzohydrazonoyl chloride 2b: Compound 2b was obtained as a colorless solid, yield 62% following the general procedure (EtOAC/Hexane 1/30). ¹H NMR (500 MHz, CDCl₃) δ 7.94 (br, NH, 1H), 7.86 (d, J=8.9 Hz, 2H), 7.31 (m, 2H), 7.16 (d, J=7.7 Hz, 2H), 6.93 (d, J=8.9 Hz, 2H), 3.85 (s, 3H), ¹³C NMR (151 MHz, CDCl₃) δ 160.53, 143.56, 132.10, 132.04, 131.89, 129.32, 128.51, 128.43, 127.88, 127.14, 124.71, 120.82, 113.77, 113.26, 55.38. HR-ESI C₁₄H₁₃N₂OCl cal. 260.0716. found. 260.0717. IR (neat) 3314, 1600, 1500, 1434, 1259, 1109, 940, 825, 754

(E/Z)—N′-(4-cyanophenyl)benzohydrazonoyl chloride 2c: Compound 2c was obtained as a yellow solid, yield 96% following the general procedure (EtOAC/Hexane 1/30). ¹H NMR (500 MHz, CDCl₃) δ 8.21 (s, NH, 1H), 8.01 (d, J=8.5 Hz, 2H), 7.68 (d, J=8.6 Hz, 2H), 7.34 (dd, J=8.6, 7.3 Hz, 2H), 7.24 (m, 2H), 7.01 (m, 1H)¹³C NMR (126 MHz, CDCl₃) δ 142.53, 138.45, 132.18, 132.13, 132.05, 129.50, 128.56, 128.46, 126.51, 122.43, 122.06, 118.60, 113.73, 112.09. HR-ESI C₁₄H₁₀N₃Cl (M+Na)+ cal. 278.0461. found 278.0454. IR (neat) 3288, 2219, 1601, 1544, 1495, 1237, 1164, 947, 833, 736

(E/Z)—N′-(4-nitrophenyl)benzohydrazonoyl chloride 2e: Compound 2e was obtained as a yellow solid, yield 45% following the general procedure (DCM). ¹H NMR (500 MHz, CDCl₃) δ 8.32 (m, 3H), 8.07 (d, J=9.0 Hz, 2H), 7.35 (dd, J=8.6, 7.3 Hz, 2H), 7.22 (dd, J=8.6, 1.1 Hz, 2H), 7.02 (m, 1H).

General Procedure C for the Preparation of Benzo-1,2,4-Triazinyl Radical 4(a-j)

A solution of 2a (1.50 g, 5.70 mmol), aniline (0.57 ml, 6.27 mmol) and TEA (1.20 ml, 8.65 mmol) in 25 mL benzene was refluxed overnight, the solvent was removed on rotavap and added 50 mL cold water, extracted with CH₂Cl₂, washed with brine, dried over MgSO4. The solvent was evaporated under reduce pressure to obtain intermediate 3a. A solution of 3a, Pd/C (9.5 mg, 1.6 mol %) and DBU (0.8 ml) in dry CH₂Cl₂ (50 ml was stirred in air at room temperature for 3 h until TLC showed the presence of a new fast running brown compound (CH₂Cl₂/hexane 1/1). The solvent was evaporated under reduced pressure, and the residue was purified with neutral alumina (Brockman I) column chromatography (CH₂Cl₂/hexane 2/1) to give product 4a as black solid.

1,3-Diphenyl-1,4-dihydro-1,2,4-benzotriazin-4-yl 4a: black solid, yield 36%. HRMS C₁₉H₁₄N₃ cal. 284.1188. found 284.1175.

1-Phenyl-3-(4-methoxyphenyl)-1,2,4-benzotriazin-4-yl 4b: black solid, yield 50%. HRMS C₂₀H₁₇N₃O (M+H)+ cal. 315.1372. found 315.1359. IR (neat) 1606, 1479, 1390, 1247, 1167, 1026, 837, 753

1-Phenyl-3-(4-cyanophenyl)-1,2,4-benzotriazin-4-yl 4c: dark green solid, yield 25%. HRMS C₂₀H₁₃N₄(M)+ cal. 309.1140. found 309.1130. IR (neat) 3044, 2227, 1592, 1482, 1385, 1207, 1079, 858, 735

7-Cyano-1,3-diphenyl-1,4-dihydro-1,2,4-benzotriazin-4-yl 4d: dark solid, yield 30%

1-Phenyl-3-(4-nitrophenyl)-1,2,4-benzotriazin-4-yl 4e: black solid, yield 58% HRMS C₁₉H₁₃N₄O₂ (M+) cal. 329.1039. found 329.1044.

2,4-diphenyl-1,4-dihydro-[1,2,4]triazino[6,5-h]quinolin-1-yl 4f: brown solid, yield 20%, ESI-MS C₂₂H₁₅N₄(M+Na)+ found 358.1302.

2,4-diphenyl-1,4-dihydronaphtho[1,2-e][1,2,4]triazin-1-yl 4g: brown solid, yield 17%, HRMS C₂₃H₁₆N₃(M+H)+ cal. 334.1344. found 334.1339.

5-methyl-1,3-diphenyl-1,4-dihydrobenzo[e][1,2,4]triazin-4-yl 4h: brown solid, yield 30%, HRMS C₂₀H₁₆N₃(M+H)+ cal. 299.1244. found 299.1407.

5-ethyl-1,3-diphenyl-1,4-dihydrobenzo[e][1,2,4]triazin-4-yl 4i: brown solid, yield 25%

5-tertbutyl-1,3-diphenyl-1,4-dihydrobenzo[e][1,2,4]triazin-4-yl 4j: brown solid, yield 21%, HRMS C₂₃H₂₂N₃(M)+ cal. 340.1814. found 340.1809.

General Procedure D for the Preparation of Triazine Unimer Compounds 5(a-j)

A solution of 4a (1.75 mmol, 500 mg) and 1-bromoethylbenzene (1.5 eq, 2.6 mmol, 0.36 ml) in benzene (10 ml) was transferred to a mixture of CuBr (2.6 mmol, 0.37 g), PMDETA (5.2 mmol, 1.09 ml), and Cu (0) (2.6 mmol, 0.17 g) in benzene (10 ml) under inert atmosphere of argon. The reaction mixture was stirred at room temperature for 24 h. The mixture was filtered off, diluted with CH₂Cl₂ and then washed with water. The organic layer was dried over anhydrous MgSO₄. The solvent was removed under reduced pressure and then the crude product was purified by silica gel column chromatography (Ethyl acetate:hexane/5:95).

### Example 1

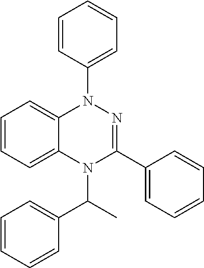 [SMILES: CC(c1ccccc1)N1C(c2ccccc2)=NN(c2ccccc2)c2ccccc21]

### 1,3-diphenyl-4-(1-phenylethyl)-1,4-dihydro-1,2,4-benzotriazine

5a: yellow powder yield 82%. ¹H NMR (500 MHz, CDCl₃) δ 7.91 (m, 2H), 7.40 (m, 3H), 7.33 (t, J=7.8 Hz, 2H), 7.24 (m, 3H), 7.19 (m, 4H), 7.13 (m, 1H), 6.84 (m, 2H), 6.75 (m, 1H), 6.52 (d, J=5.0 Hz, 1H), 4.66 (q, J=7.0 Hz, 1H), 1.75 (d, J=7.1 Hz, 3H). ¹³C NMR (151 MHz, CDCl₃) δ 149.42, 144.02, 143.28, 141.46, 134.52, 130.44, 129.30, 128.84, 128.40, 127.91, 127.63, 127.56, 127.49, 125.08, 124.45, 124.40, 123.28, 122.50, 111.88, 61.15, 19.70. HR-ESI: C₂₇H₂₃N₃ cal. 389.1892. found 389.1900. IR (neat) 2982, 1586, 1486, 1293, 1053, 757

### Example 2

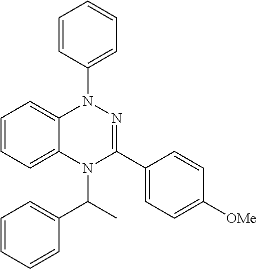 [SMILES: COc1ccc(C2=NN(c3ccccc3)c3ccccc3N2C(C)c2ccccc2)cc1]

### 1-Phenyl-3-(4-methoxyphenyl)-4-(1-phenylethyl)-1,4-dihydro-1,2,4-benzotriazine

5b: yellow powder, yield 58%. ¹H NMR (500 MHz, CDCl₃) δ 7.84 (d, J=9.0 Hz, 2H), 7.32 (dd, J=8.4, 7.2 Hz, 2H), 7.24 (m, 3H), 7.19 (m, 4H), 7.11 (t, J=7.3 Hz, 1H), 6.94 (m, 2H), 6.83 (m, 2H), 6.74 (m, 1H), 6.53 (m, 1H), 4.68 (q, J=7.1 Hz, 1H), 3.86 (s, 3H), 1.75 (d, J=7.1 Hz, 3H). ¹³C NMR (126 MHz, DMSO-d₆) δ 160.93, 150.24, 143.70, 143.32, 141.80, 130.89, 129.44, 129.30, 128.36, 127.87, 127.48, 126.15, 125.61, 124.70, 124.22, 123.09, 122.83, 114.53, 111.78, 60.76, 55.72, 20.38. HRMS C₂₈H₂₆N₅O₃ (M+H)+ Cal 420.2076, found 420.2057. IR (neat) 2832, 1602, 1452, 1252, 1166, 1038, 841, 737

### Example 3

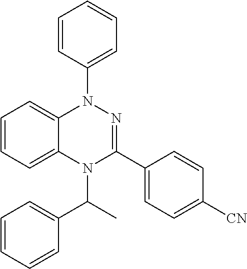 [SMILES: CC(c1ccccc1)N1C(c2ccc(C#N)cc2)=NN(c2ccccc2)c2ccccc21]

### 1-Phenyl-3-(4-cyanophenyl)-4-(1-phenylethyl)-1,4-dihydro-1,2,4-benzotriazine

5c: orange powder, yield 71%. ¹H NMR (500 MHz, CDCl₃) δ 7.99 (d, J=8.4 Hz, 2H), 7.65 (d, J=8.4 Hz, 2H), 7.33 (m, 2H), 7.28-7.14 (m, 4H), 7.12-7.04 (m, 4H), 6.96-6.81 (m, 3H), 6.48 (dd, J=8.0, 1.4 Hz, 1H), 4.47 (q, J=7.1 Hz, 1H), 1.73 (d, J=7.1 Hz, 3H), ¹³C NMR (126 MHz, CDCl₃) δ 146.22, 143.80, 142.69, 140.81, 139.55, 132.20, 129.53, 128.98, 128.00, 127.85, 127.61, 127.59, 125.60, 125.30, 125.30, 123.76, 123.33, 118.91, 112.16, 112.14, 62.38, 19.73. HRMS C₂₈H₂₂N₄ (M+H)+ Cal 415.1923. found 415.1906. IR (neat) 2930, 2224, 1588, 1485, 1293, 846, 755

### Example 4

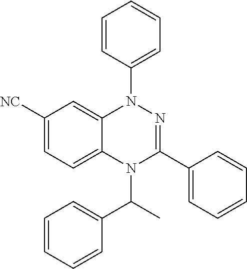 [SMILES: CC(c1ccccc1)N1C(c2ccccc2)=NN(c2ccccc2)c2cc(C#N)ccc21]

### 1,3-diphenyl-4-(1-phenylethyl)-1,4-dihydrobenzo[e][1,2,4]triazine-7-carbonitrile

5d: Yellow solid, yield 61%, 1H NMR (500 MHz, CDCl₃) δ 7.75 (m, 2H), 7.43-7.36 (m, 5H), 7.28 (m, 3H), 7.24-7.17 (m, 5H), 7.03 (dd, J=8.0, 1.8 Hz, 1H), 6.64 (d, J=8.1 Hz, 1H), 6.56 (d, J=1.8 Hz, 1), ¹³C NMR (126 MHz, CDCl₃) δ 149.9, 142.4, 140.6, 136.2, 133.4, 129.8, 129.4, 128.5, 128.3, 127.9, 127.8, 127.2, 126.7, 125.8, 123.7, 122.8, 118.9, 114.1, 108.2, 60.9, 19.6. HRMS C₂₈H₂₂N₄(M+Na)+ cal. 437.1744. found 437.1753.

### Example 5

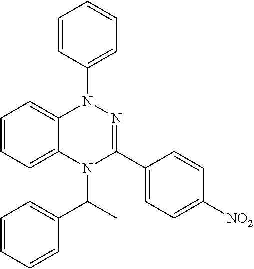 [SMILES: CC(c1ccccc1)N1C(c2ccc([N+](=O)[O-])cc2)=NN(c2ccccc2)c2ccccc21]

### 3-(4-nitrophenyl)-1-phenyl-4-(1-phenylethyl)-1,4-dihydrobenzo[e][1,2,4]triazine

5e: Orange solid, 75%, 1H NMR (500 MHz, CDCl3) δ 8.23 (d, J=8.8 Hz, 2H), 8.06 (d, J=8.8 Hz, 2H), 7.35 (t, J=7.8 Hz, 2H), 7.29-7.16 (m, 4H), 7.11 (d, J=7.4 Hz, 2H), 7.08 (d, J=7.4 Hz, 2H), 6.95 (td, J=7.5, 1.4 Hz, 1H), 6.91-6.85 (m, 2H), 6.50 (d, J=8.0 Hz, 1H), 4.48 (q, J=7.1 Hz, 1H), 1.75 (d, J=7.1 Hz, 3H), ¹³C NMR (126 MHz, CDCl₃) δ 147.9, 145.7, 143.7, 142.6, 141.5, 140.7, 129.4, 129.0, 128.0, 127.9, 127.7, 127.6, 125.6, 125.5, 125.4, 123.8, 123.7, 123.5, 112.2, 62.6, 19.7. HRMS C₂₇H₂₂N₄O₂ (M+Na)+ cal. 457.1640. found 457.1638.

### Example 6

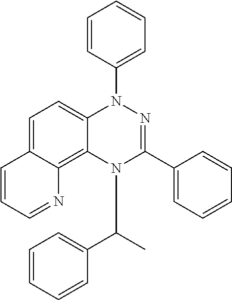 [SMILES: CC(c1ccccc1)N1C(c2ccccc2)=NN(c2ccccc2)c2ccc3cccnc3c21]

### 2,4-diphenyl-1-(1-phenylethyl)-1,4-dihydro-[1,2,4]triazino[6,5-h]quinoline

5f: Orange solid, yield 64%, %, 1H NMR (500 MHz, CDCl3) δ 8.99 (m, 1H), 7.98 (d, J=7.9 Hz, 2H), 7.49-6.95 (m, 17H), 5.27 (m, 1H), 1.64 (br, 3H), ¹³C NMR (126 MHz, CDCl₃) δ 150.2, 135.8, 129.3, 129.1, 128.9, 128.6, 128.5, 128.4, 128.1, 128.0, 127.5, 127.4, 127.3, 127.2, 127.1, 126.6, 126.0, 125.4, 124.8, 124.4, 122.4, 121.6, 114.6, 63.6, 21.1, HR-ESI C₃₀H₂₄N₄(M)+ cal. 440.2001. found 440.1991.

### Example 7

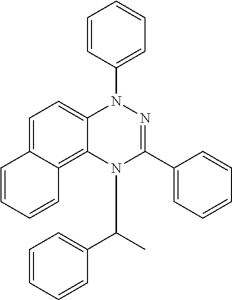 [SMILES: CC(c1ccccc1)N1C(c2ccccc2)=NN(c2ccccc2)c2ccc3ccccc3c21]

### 2,4-diphenyl-1-(1-phenylethyl)-1,4-dihydronaphtho[1,2-e][1,2,4]triazine

5g: Yellow solid, yield 38%, 1H NMR (500 MHz, CDCl₃, both diastereomers) δ 8.57 (d, J=8.5 Hz, 1H, minor diastereomer), 8.25-8.15 (m, 5H), 7.78 (m, 2H), 7.59-7.33 (m, 16H), 7.22-7.10 (m, 12H), 7.09-6.90 (m, 6H), 4.70 (q, J=7.1 Hz, 1H, minor diastereomer), 4.63 (q, J=7.3 Hz, 1H, major diastereomer), 1.71 (d, J=7.3 Hz, 3H, minor diastereomer), 1.58 (d, J=7.3 Hz, 3H, major diastereomer), ¹³C NMR (126 MHz, CDCl₃, both diastereomer) δ 137.3, 134.9, 131.5, 131.1, 129.3, 129.1, 129.0, 128.7, 128.6, 128.5, 128.3, 128.2, 128.1, 128.0, 127.8, 127.6, 127.5, 127.4, 127.3, 127.1, 127.0, 126.5, 126.4, 126.0, 125.8, 125.5, 124.6, 123.8, 123.1, 122.8, 122.2, 120.9, 117.0, 116.5, 113.9, 113.7, 64.8, 64.3, 20.7, 20.0, HR-ESI C₃₁H₂₅N₃ (M+H)+ cal. 440.2127. found 440.2117.

### Example 8

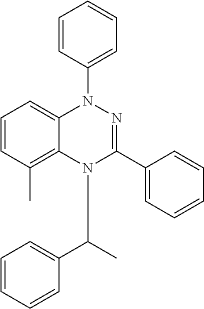 [SMILES: Cc1cccc2c1N(C(C)c1ccccc1)C(c1ccccc1)=NN2c1ccccc1]

### 5-methyl-1,3-diphenyl-4-(1-phenylethyl)-1,4-dihydrobenzo[e][1,2,4]triazine

5h: Yellow sticky oil, yield 29%, 1H NMR (500 MHz, CDCl₃, both diastereomers) δ 8.07-7.92 (m, 4H), 7.50-7.09 (m, 26H), 7.01-6.80 (m, 4H), 6.60 (d, J=8.2 Hz, 1H, minor diastereomer), 6.46 (d, J=8.3 Hz, 1H, major diastereomer), 4.58 (m, 1H, minor diastereomer), 4.45 (m, 1H, major diastereomer), 2.51 (s, 3H, minor diastereomer), 2.34 (s, 3H, major diastereomer), 1.68 (d, J=7.0 Hz, 3H, minor diastereomer), 1.49 (d, J=7.5 Hz, 3H, major diastereomer), ¹³C NMR (126 MHz, CDCl₃, both diastereomer) δ 147.6, 144.9, 143.2, 141.6, 137.2, 134.0, 130.7, 128.9, 128.7, 128.2, 128.1, 127.7, 127.6, 127.1, 125.5, 125.3, 125.1, 124.9, 124.6, 123.4, 109.9, 109.7, 65.0, 63.5, 19.9, 19.5, 18.0, 16.9, HR-ESI C₂₈H₂₅N₃ (M+Na)+ cal. 426.1946. found 426.1941.

### Example 9

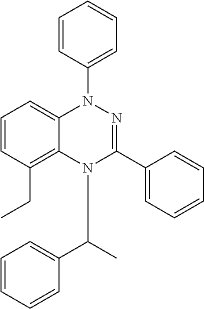 [SMILES: CCc1cccc2c1N(C(C)c1ccccc1)C(c1ccccc1)=NN2c1ccccc1]

### 5-ethyl-1,3-diphenyl-4-(1-phenylethyl)-1,4-dihydrobenzo[e][1,2,4]triazine

5i: Yellow sticky oil, yield 24%. 1H NMR (500 MHz, CDCl₃, both diastereomers) δ 8.03 (dd, J=7.6, 2.0 Hz, 4H, major diastereomer), 7.96 (dd, J=6.7, 3.0 Hz, 4H, minor diastereomer), 7.46-6.86 (m, 30H), 6.62 (dd, J=8.1, 1.4 Hz, 1H, minor diastereomer), 6.44 (m, 1H, major diastereomer), 4.55 (q, J=7.0 Hz, 1H, minor diastereomer), 4.40 (q, J=7.3 Hz, 1H, major diastereomer), 3.18 (m, 1H, minor diastereomer), 3.00 (m, 1H, major diastereomer), 2.75 (m, 1H, minor diastereomer), 2.65 (m, 1H, major diastereomer), 1.65 (d, J=7.0 Hz, 3H, minor diastereomer), 1.47 (d, J=7.2 Hz, 3H, major diastereomer), 1.37 (t, J=7.6 Hz, 3H, major diastereomer), 1.30 (t, J=7.6 Hz, 3H, minor diastereomer), ¹³C NMR (126 MHz, CDCl₃, both diastereomer) δ 147.1, 144.8, 143.2, 141.5, 139.9, 137.5, 130.0, 128.9, 128.8, 128.7, 128.6, 128.5, 128.2, 128.1, 127.6, 127.4, 127.2, 127.0, 125.4, 125.1, 124.6, 123.5, 123.4, 123.3, 123.2, 116.6, 116.0, 109.7, 109.5, 66.0, 63.7, 23.4, 22.8, 20.2, 19.4, 14.9, 14.7. ESI-MS C₂₉H₂₇N₃ (M+Na)+ cal. 440.21. found 440.21.

### Example 10

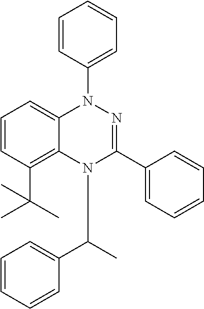 [SMILES: CC(c1ccccc1)N1C(c2ccccc2)=NN(c2ccccc2)c2cccc(C(C)(C)C)c21]

### 5-tert-butyl-1,3-diphenyl-4-(1-phenylethyl)-1,4-dihydrobenzo[e][1,2,4]triazine

5j: sticky oil, yield 26%. 1H NMR (500 MHz, CDCl₃, both diastereomers) δ 8.18 (m, 2H, major diastereomer), 8.07 (m, 2H, minor diastereomer), 7.58-7.15 (m, 18H), 7.06-6.73 (m, 14H), 4.76 (q, J=7.0 Hz, 1H, major diastereomer), 4.66 (q, J=6.9 Hz, 1H, minor diastereomer), 1.85 (d, J=7.1 Hz, 3H, major diastereomer), 1.68 (s, 9H, minor diastereomer), 1.48 (s, 9H, major diastereomer), 1.25 (d, J=6.9 Hz, 3H, minor diastereomer). ¹³C NMR (126 MHz, CDCl₃) δ 157.4, 157.0, 151.3, 150.4, 145.5, 144.6, 140.5, 140.3, 137.9, 137.5, 136.2, 136.0, 135.7, 134.5, 130.9, 130.8, 129.1, 129.1, 128.9, 128.8, 128.7, 128.6, 128.5, 128.4, 128.3, 128.2, 128.0, 127.6, 127.4, 127.3, 125.6, 124.9, 123.6, 123.1, 122.7, 122.6, 122.0, 121.9, 116.6, 116.0, 62.0, 60.3, 36.1, 35.8, 30.9, 30.8, 19.1, 17.43. HR-ESI C₃₁H₃₁N₃ (M+H)+ cal. 446.2596. found 446.

### Example 11

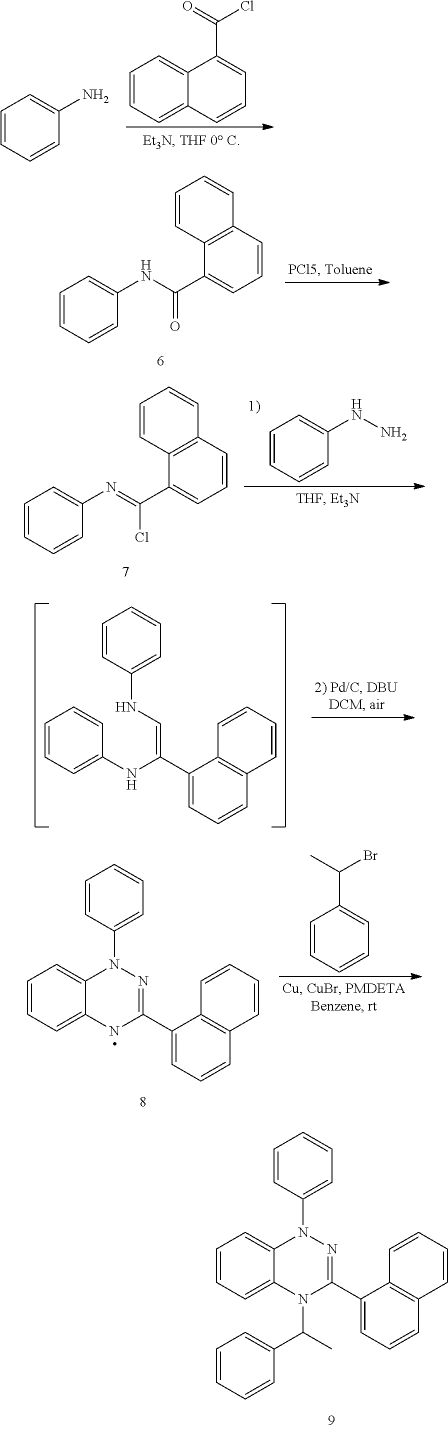

N-phenyl-1-naphthamide 6: Triethylamine (4.4 mL, 31 mmol) was added to a solution of aniline (2.6 mL, 28 mmol) in THF (50 mL) at 0° C. 1-Naphthanoyl chloride (5 g, 26 mmol) in THF (20 mL) was added dropwise. The reaction mixture was then stirred for 18 h and was slowly warmed to room temperature. Then the solvent was evaporated under reduced pressure and the residue was dissolved in ethyl acetate (150 ml), washed with water (2×100 ml) and dried over MgSO₄. The solvent was removed in vacuo. Recrystallization from minimum amount of dichloromethane gave rise to 6. (6 g, 92%). ¹H NMR (500 MHz, CDCl₃) δ 8.39 (m, 1H), 7.96 (d, J=8.3 Hz, 1H), 7.93 (m, 1H), 7.76-7-67 (m, 4H), 7.59-7.54 (m, 2H), 7.49 (dd, J=8.3, 7.0 Hz, 1H), 7.40 (m, 2H), 7.18 (m, 1H). EI-MS C17H13NO found 247.10.

(E/Z)—N-phenyl-1-naphthimidoyl chloride 7: An equimolar mixture of the corresponding amide (2 g, 8 mmol) with PCl₅ (1.68 g, 8 mmol) was heated in toluene under reflux for 4 h. Afterward, the resulting mixture was allowed to cool to room temperature, the solvent was evaporated under reduced pressure. The products obtained were used in the next step without further purification.

8: Triethylamine (1.7 mL, 12 mmol) was added to a solution of phenylhydrazine (0.8 mL, 8 mmol) in THF (50 mL) at 0° C. Compound 7 (8 mmol) in THF (10 mL) was added dropwise. The reaction mixture was then stirred for 18 h and was slowly warmed to room temperature. Then the solvent was evaporated under reduced pressure and the residue was dissolved in ethyl acetate (100 ml), washed with water (2×50 mL) and dried over MgSO₄. The solvent was removed in vacuo. The mixture was treated with Pd/C (1.6 mol %) and DBU (1 eq) in dry CH₂Cl₂ (50 mL). The reaction mixture was stirred in air at room temperature for 3 h until TLC showed the presence of a new fast running brown compound (CH₂Cl₂/hexane 1/1). The solvent was evaporated under reduced pressure, and the residue was purified with neutral alumina (Brockman I) column chromatography (CH₂Cl₂/hexane 2/1) to give product 8 (26% yield) as black solid.

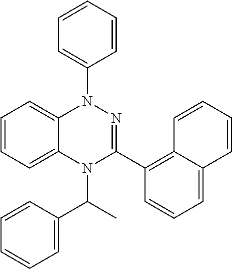 [SMILES: CC(c1ccccc1)N1C(c2cccc3ccccc23)=NN(c2ccccc2)c2ccccc21]

### 3-(naphthalen-1-yl)-1-phenyl-4-(1-phenylethyl)-1,4-dihydrobenzo[e][1,2,4]triazine

9: General procedure C was followed to give yellow solid, 26% 1H NMR (500 MHz, CDCl3) δ 8.35 (m, 1H), 7.84 (dd, J=8.1, 1.0 Hz, 2H), 7.58-7.31 (m, 8H), 7.29-7.08 (m, 6H), 6.79 (m, 2H), 6.57 (m, 2H), 4.75 (q, J=7.0 Hz, 1H), 1.55 (d, J=7.6 Hz, 3H), ¹³C NMR (126 MHz, CDCl₃) δ 150.2, 144.1, 141.9, 141.6, 133.7, 133.4, 132.2, 131.5, 129.7, 129.1, 128.4, 128.3, 128.3, 127.1, 126.9, 126.7, 126.1, 125.7, 125.1, 124.2, 124.1, 122.8, 122.6, 118.9, 112.4, 58.3, 19.3. HR-ESI C₃₁H₂₅N₃ (M+H)+ cal. 440.2127. found 440.2134.

### Example 12

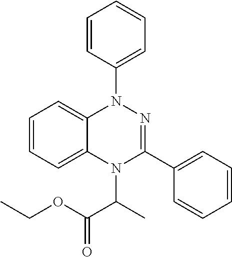 [SMILES: CCOC(=O)C(C)N1C(c2ccccc2)=NN(c2ccccc2)c2ccccc21]

### ethyl 2-(1,3-diphenylbenzo[e][1,2,4]triazin-4(1H)-yl)propanoate

General procedure D was followed, and ethyl 2-bromopropanoate was used as alkylbromide. Brown sticky oil, yield 14%, 1H NMR (500 MHz, CDCl3) δ 7.75 (m, 2H), 7.50-7.31 (m, 6H), 7.17-7.06 (m, 2H), 6.97 (m, 1H), 6.83 (m, 2H), 6.57 (m, 1H), 4.21 (m, 3H), 1.58 (d, J=7.2 Hz, 3H), 1.27 (t, J=7.1 Hz, 3H)

### Example 13

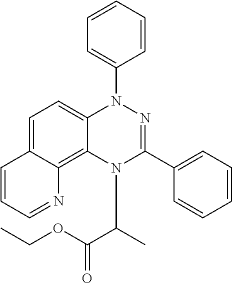 [SMILES: CCOC(=O)C(C)N1C(c2ccccc2)=NN(c2ccccc2)c2ccc3cccnc3c21]

### ethyl 2-(2,4-diphenyl-[1,2,4]triazino[6,5-h]quinolin-1 (4H)-yl)propanoate

General procedure D was followed, and ethyl 2-bromopropanoate was used as alkylbromide. Orange solid, yield 25%, 1H NMR (500 MHz, CDCl3) δ 8.79 (d, J=2.4 Hz, 1H), 8.06 (dd, J=7.3, 2.3 Hz, 2H), 7.95 (dd, J=8.2, 1.8 Hz, 1H), 7.55 (m, 2H), 7.49-7.34 (m, 6H), 7.20 (m, 2H), 7.10 (d, J=8.9 Hz, 1H), 4.62 (q, J=7.1 Hz, 1H), 4.10 (m, 2H), 1.45 (d, J=7.1 Hz, 3H), 1.18 (t, J=7.2 Hz, 3H). HRMS C₂₇H₂₄N₄O₂ (M+Na)+ cal. 459.1797. found 459.1809.

### Example 14

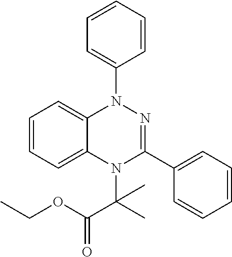 [SMILES: CCOC(=O)C(C)(C)N1C(c2ccccc2)=NN(c2ccccc2)c2ccccc21]

### ethyl 2-(1,3-diphenylbenzo[e][1,2,4]triazin-4(1H)-yl)-2-methylpropanoate

General procedure D was followed, and ethyl 2-bromo-2-methylpropanoate was used as alkylbromide. Brown sticky oil, yield 78%, ¹H NMR (500 MHz, CDCl₃) δ 7.98 (m, 2H), 7.56 (dd, J=8.6, 1.2 Hz, 2H), 7.44 (dd, J=8.5, 7.3 Hz, 2H), 7.36 (m, 3H), 7.20 (m, 2H), 7.01 (td, J=7.5, 1.5 Hz, 1H), 6.96 (td, J=7.8, 1.6 Hz, 1H), 6.84 (dd, J=8.0, 1.5 Hz, 1H), 4.12 (q, J=7.2 Hz, 2H), 1.47 (s, 3H), 1.28 (t, J=7.0 Hz, 3H). HRMS C₂₅H₂₅N₃O₂ (M+Na)+ cal. 422.1844. found 422.1841.

### Example 15-19

The following compounds are prepared essentially according to the procedures and examples set forth above, with modifications where necessary of the starting materials to provide the desired product.

| Example | | |
|---|---|---|
| No. | Compound | |
| 15 | 3-mesityl-1-phenyl- 4-(1-phenylethyl)-1,4- dihydrobenzo [e][1,2,4]triazine | 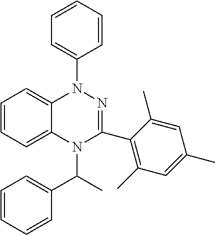 [SMILES: Cc1cc(C)c(C2=NN(c3ccccc3)c3ccccc3N2C(C)c2ccccc2)c(C)c1] |
| 16 | 5-tert-butyl-3- mesityl- 1-phenyl-4-(1- phenylethyl)-1,4- dihydrobenzo [e][1,2,4]triazine | 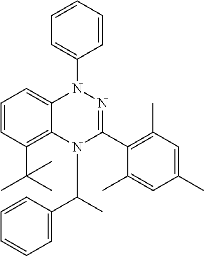 [SMILES: Cc1cc(C)c(C2=NN(c3ccccc3)c3cccc(C(C)(C)C)c3N2C(C)c2ccccc2)c(C)c1] |
| 17 | 5-isopropyl-1,3- diphenyl-4- (1-phenylethyl)- 1,4-dihydrobenzo [e][1,2,4]triazine | 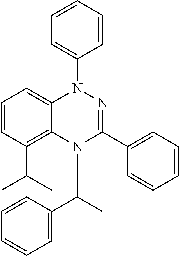 [SMILES: CC(C)c1cccc2c1N(C(C)c1ccccc1)C(c1ccccc1)=NN2c1ccccc1] |
| 18 | (4-(1-(1,3- diphenylbenzo [e][1,2,4]triazin- 4(1H)-yl)ethyl) phenyl)methanol | 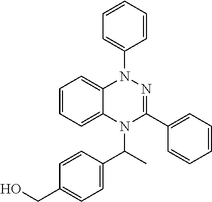 [SMILES: CC(c1ccc(CO)cc1)N1C(c2ccccc2)=NN(c2ccccc2)c2ccccc21] |
| 19 | 7,9-diphenyl-10- (1-phenylethyl)-7,10- dihydropyreno [1,2-e][1,2,4]triazine | 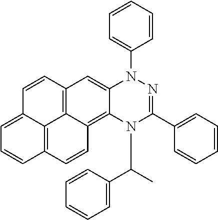 [SMILES: CC(c1ccccc1)n1c(-c2ccccc2)nn(-c2ccccc2)c2cc3ccc4cccc5ccc(c3c45)c21] |

### Example 20

### General Procedure for Styrene Polymerization

A vial equipped with a magnetic stir bar and fitted with a teflon screw cap septum was charged with a desired compound of Example 1-19 (10 mg, 0.025 mmol, 1 eq) and styrene (0.74 ml, 6.4 mmol, 250 eq). The solution was degassed using three freeze-pump-thaw cycles. The vial was then backfilled with argon and stirred at 125° C. for 6 h. The reaction mixture was dissolved in dichloromethane (1 ml) and precipitated in MeOH. The resulting solid was dried, re-dissolved, and precipitated a second time into MeOH. After drying, the polymers were analyzed by GPC to give the number average molecular weight (Mₙ), weight average molecular weight (Mw) and molecular weight distribution (M_{w}/Mₙ) of the polymer. PDI=polydispersity index.

### Example 21

### Comparative Example

Triazine radicals 4a-c were used to mediate the polymerization of styrene in the bulk (see Table 1). When heating only the triazine radicals with styrene no monomer conversion was detected over the first 3 hours, but a gradual increase in molecular weight was observed after this induction period. Similarly, when 4a was heated to 125° C. in the presence of the thermal radical initiator, benzoyl peroxide (BPO) (molar ratio 1:0.5), and styrene, there was an induction period of around 2 hours before polymerization initiated, eventually reaching 28% monomer conversion after 7 h. The resulting polymer had relatively low polydispersities, indicating the potential of the radical for mediating polymerization. However, a significant deviation was observed between the experimental and theoretical molecular weights, suggesting that further refinement of the system was necessary to access targeted polymer properties. The structures of Compounds 4a and 4c are shown in Table 1 below:

TABLE 1
Polymerization of styrene (250 eq) at
125° C. in bulk mediated by triazinyl radical 4a and 4c

| | Reaction | Time | % | Mₙ^{a} | Mₙₜₕ^{b} | |
|---|---|---|---|---|---|---|
| Entry | Conditions | (h) | Conversion^{c} | (g/mol) | (g/mol) | PDI |
| 1 | 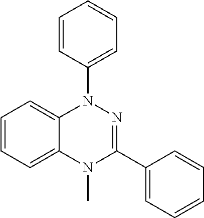 [SMILES: CN1C(c2ccccc2)=NN(c2ccccc2)c2ccccc21] 4a^{d}, BPO (0.5 eq) | 1 2 3 4 5 7 9 12 | n n 14 17 23 29 31 36 | n n 4200 9200 11200 15100 16000 16300 | n n 3600 4400 5900 7600 8000 9500 | n n 1.16 1.24 1.35 1.53 1.65 1.68 |
| 2 | 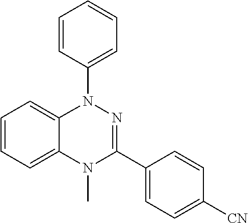 [SMILES: CN1C(c2ccc(C#N)cc2)=NN(c2ccccc2)c2ccccc21] 4c | 1 3 5 7 9 11 13 | n n 3 12 20 22 22 | n n 2600 5400 7800 7700 7900 | n n 800 3000 5300 5800 5800 | n n 1.08 1.14 1.33 1.42 1.53 |
| 3 | 4c, BPO (0.5 eq) | 1 | n | n | n | n |
| | | 3 | n | n | n | n |
| | | 5 | 7 | 5400 | 1800 | 1.17 |
| | | 7 | 12 | 8300 | 3100 | 1.44 |
| | | 10 | 15 | 9100 | 4000 | 1.54 |
| | | 12 | 20 | 10000 | 5200 | 1.61 |

^{a}Determined by GPC analysis;
^{b}theoretical molecular weight calculated on the basis of monomer conversion,
^{c}conversion determined by ¹H NMR,
^{d}U.S. Pat. No. 3,423,409,
n = not measurable.

### Example 22

### Styrene Polymerization

Examples 1-3 were polymerized according to the procedure in Example 20. The polymerization of styrene proceeded in a controlled manner, showing a good correlation between experimental and theoretical molecular weights while maintaining PDIs in a range from 1.1-1.3 (Table 2).

TABLE 2
Polymerization of styrene (250 eq) at
125° C. in bulk mediated Example 1-3

| | Reaction | % Conver- | Mₙ^{a} | Mₙₜₕ^{b} | |
|---|---|---|---|---|---|
| Entry | Conditions | sion^{c} | (kg/mol) | (kg/mol) | PDI |
| 1 | Ex. 1, 1 h | 23 | 5.4 | 6.0 | 1.15 |
| 2 | Ex. 1, 2 h | 33 | 8.3 | 8.7 | 1.15 |
| 3 | Ex. 1, 4 h | 50 | 14.8 | 13.0 | 1.18 |
| 4 | Ex. 1, 8 h | 67 | 20.4 | 17.4 | 1.23 |
| 5 | Ex. 2, 2 h | 15 | 2.3 | 3.9 | 1.20 |
| 6 | Ex. 2, 4 h | 27 | 6.3 | 7.0 | 1.14 |
| 7 | Ex. 2, 6 h | 49 | 10.8 | 12.8 | 1.17 |
| 8 | Ex. 2, 8 h | 58 | 13.4 | 15.1 | 1.21 |
| 9 | Ex. 3, 2 h | 21 | 5.3 | 5.6 | 1.15 |
| 10 | Ex. 3, 4 h | 39 | 8.0 | 10.1 | 1.16 |
| 11 | Ex. 3, 6 h | 47 | 10.0 | 12.2 | 1.21 |
| 12 | Ex. 3, 8 h | 52 | 11.1 | 13.5 | 1.28 |

^{a}Determined by GPC analysis,
^{b}theoretical molecular weight calculated from monomer conversion,
^{c}conversion determined by ¹H NMR.

Different molecular weights were targeted for polystyrene by simply adjusting reaction parameters. Molecular weights from 1-40 kg/mol were targeted for Example 3 with good agreement between experimental and theoretical molecular weight, while maintaining very low polydispersities (PDI<1.3). This demonstrates the ability to access well-defined polymer structures using for triazine-mediated polymerization (TMP) (see Table 3).

TABLE 3
Polymerization of Styrene targeted at different molecular
weight, 125° C., 6 h initiated with Example 3

| | | % conver- | | | |
|---|---|---|---|---|---|
| Entry | [Sty]₀/[Ex. 3]₀ | sion^{c} | Mn^{a} | Mnₜₕ^{b} | PDI |
| 1 | 50/1 | 22 | 1.6k | 1.16k | 1.25 |
| 2 | 100/1 | 36 | 4.2k | 3.8k | 1.20 |
| 3 | 200/1 | 45 | 7.9k | 9.4k | 1.21 |
| 4 | 300/1 | 48 | 12.5k | 15.0k | 1.18 |
| 5 | 400/1 | 53 | 19.4k | 22.2k | 1.25 |
| 3 | 600/1 | 48 | 22.3k | 30.2k | 1.33 |
| 4 | 800/1 | 56 | 35.9k | 46.9k | 1.20 |

^{a}Determined by GPC analysis,
^{b}theoretical molecular weight calculated from monomer conversion,
^{c}conversion determined by ¹H NMR.

Styrene polymerizations were also tested at lower temperatures. Polymerization of styrene (250 eq) with Example 1 at 110° C. reached 47% conversion after 22 h led to a well-defined polymer with controlled molecular weights and low polydispersities (Mn=11.8 kg/mol, Mnₜₕ=12.2 kg/mol, PDI 1.1).

The existence of “living” chain ends is a necessity in controlled radical polymerizations, and, in order to verify this for polymerizations mediated by compounds of formula I or II, a low molecular weight polystyrene sample was prepared using Example 2 and analyzed by ¹H NMR (Mₙ (NMR)=2.2 kg/mol, Mₙ (GPC)=2.3 kg/mol PDI=1.21). Moreover, the results of Gel Permeation Chromatography (GPC) demonstrated incorporation of the triazine unimer (i.e., the compounds of the invention where R³ is —CH(CH₃)(Ph)) at the polymer chain end to give the compounds of the invention where R³ is polystyrene.

### Example 23

### Block Copolymer Synthesis

A polystyrene macroinitiator synthesized using Example 1 was isolated (Mₙ=13 kg/mol, PDI=1.16) and resubmitted to the reaction conditions to extend the chain length, yielding poly(styrene)-b-(styrene) (Mₙ=25.1 kg/mol, PDI=1.26). As evidenced by little tailing in the low molecular weight region of the GPC, triazine end-groups were efficiently retained during isolation of the macroinitiator. Similarly, a diblock copolymer of styrene and 4-methoxystyrene was synthesized to verify the ability of compounds of formula I or II to produce block copolymers with other monomers. An efficient chain end capping and re-initiation occurred, demonstrating that compounds of formula I or II provide a simple-to-use method for synthesis of block copolymers. A polystyrene macroinitiator synthesized using Example 1 was isolated (Mₙ=11.9 kg/mol, PDI=1.16), and resubmitted to reaction conditions with 4-methoxystyrene to give poly(styrene)-b-(4-methoxystyrene) by chain extension (Mₙ=28.5 kg/mol, PDI=1.27).

### Example 24

### Random Copolymerization

Example 1 was used to control the polymerization of other monomer families in random copolymerizations between styrene and either methyl methacrylate or butyl acrylate (Table 4). Well-defined random copolymers of styrene and butyl acrylate were obtained with PDIs between 1.2 and 1.32. The copolymerization of styrene with methyl methacrylate readily produced well-defined random copolymers with PDIs in the range from 1.1 to 1.34. Importantly, no peaks were observed in the 5.50-6.20 ppm region of the ¹H NMR spectra, indicating that there was little or no termination by disproportionation, a key difference from NMP.

Polydispersity and molecular weights (Mn) for the various ratios of butyl acrylate and methyl methacrylate to styrene for bulk random copolymerization are listed in Table 4

TABLE 4
Bulk random copolymerization
(200 eq. of monomer), Ex. 1 at 125° C., 8 h.

| | Ratio of sty/ | Mₙ | |
|---|---|---|---|
| Co-monomer | co-monomer | (kgM/mol) | PDI |
| 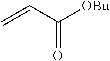 | 90/10 80/20 60/40 50/50 | 16.8k 14.8k 15.6k 14.8k | 1.17 1.18 1.27 1.32 |
| 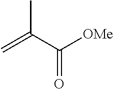 | 90/10 60/40 40/60 20/80 10/90 | 11.6k 16.4k 16.9k 14.4k 11.1k | 1.11 1.16 1.22 1.27 1.34 |

### Example 25

### Styrene Polymerization

Additional polymerization results are shown in Tables 5-10

TABLE 5
Polymerization of styrene (250 eq) at 125° C. except where stated

| | Reaction | | | | % conver- |
|---|---|---|---|---|---|
| Entry | Conditions | Mn^{a} | Mnₜₕ^{b} | PDI | sion^{c} |
| 1 | Ex. 1, bulk, 5 h | 13.1k | 12.4k | 1.16 | 49 |
| 2 | Ex. 2, bulk, 5 h | 11.1k | 12.4k | 1.10 | 47 |
| 3 | Ex. 4, bulk, 5 h | 12.9k | 12.0k | 1.15 | 46 |
| 4 | Ex. 3, bulk, 5 h | 10.2k | 13.4k | 1.21 | 51 |
| 5 | Ex. 6, bulk, 5 h | 20.9k | 18.2k | 1.55 | 70 |
| 6 | Ex. 8, bulk, 5 h | 10.5k | 12.6k | 1.10 | 48 |
| 7 | Ex. 5, bulk, 5 h | 5.2k | 8.7k | 1.31 | 33 |
| 8 | Ex. 7, bulk, 6 h | n | n | n | <5 |
| 9 | Ex. 7, bulk, 15 h | 1.6k | 7.1k | 1.88 | 27 |
| 10 | Ex. 12, bulk, 6 h | 22k | 13.2k | 1.45 | 51 |
| 11 | Ex. 13, bulk, 6 h | 17.5k | 14.5k | 1.58 | 56 |
| 12 | Ex. 9, bulk, 6 h | 10.3k | 11.3k | 1.12 | 44 |
| 13 | Ex. 10, bulk, 6 h | 15.4k | 14.0k | 1.16 | 54 |
| 14 | Ex. 11, bulk, 6 h | 12.7k | 15.9k | 1.18 | 61 |
| 15 | Ex. 1, bulk, 5 h | 13.3k | 14.6k | 1.18 | 52 |
| 16 | Ex. 1, 50% anisol, 5 h | 5.9k | 4.9k | 1.16 | 19 |
| 17 | Ex. 1, 50% DMF, 5 h | 8.8k | 4.4k | 1.39 | 17 |
| 18 | Ex. 1, 50% NMP, 5 h | 12k | 8.3k | 1.19 | 32 |
| 19 | Ex. 1, bulk, 110° C., 22.5 h | 11.8k | 12.2k | 1.05* | 47 |
| 20 | Ex. 1, bulk, 100° C., 22 h | 4.6k | 2.7k | 1.28 | 11 |
| 21 | Ex. 7, 6 h, 135° C. | 28k | 18.9k | 2.9 | 73 |
| 22 | Ex. 7, 15 h, 145° C. | 97k | 18.9k | 2.47 | 73 |
| 23 | Ex. 6, bulk, 110° C., 7 h | 8.8 | 6.0 | 1.26 | 24 |

^{a}Determined by GPC analysis,
^{b}theoretical molecular weight calculated from monomer conversion,
^{c}conversion determined by ¹H NMR,
n = not measurable.

TABLE 6
Co-polymerization of methyl methacrylate (200 eq) with styrene

| | Reaction | Sty/ | | | | % conver- |
|---|---|---|---|---|---|---|
| Entry | Cond. | Co-monomer | Mn^{a} | Mnₜₕ^{b} | PDI | sion^{c} |
| 1 | Ex. 10, 6 h | 90/10 | 15.9 | 10.6 | 1.15 | |
| 2 | Ex. 10, 6 h | 10/90 | 17.1 | 9.8 | 1.32 | |
| 3 | Ex. 10, 6 h | 5/95 | 25.8 | | 1.37 | |
| 4 | Ex. 10, 6 h | 1/99 | 38.3 | | 1.48 | |
| 5 | Ex. 9, 6 h | 10/90 | 13.2 | 8.0 | 1.39 | |

^{a}Determined by GPC analysis,
^{b}theoretical molecular weight calculated from monomer conversion,
^{c}conversion determined by ¹H NMR.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

1. A compound of formula: 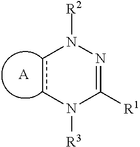
or an acceptable salt thereof, wherein
the dashed line represents an optional double bond;
A is selected from aryl and heteroaryl, each of which is independently optionally substituted with one or more R⁴;
wherein each R⁴ is independently selected from the group consisting of halogen, —NO₂, —CN, C₁-C₂₀ alkyl, C₂-C₂₀ alkenyl, C₂-C₂₀ alkynyl optionally substituted with —Si(C₁-C₆ alkyl)₃, C₁-C₂₀ haloalkyl, —OH, C₁-C₂₀ alkoxy, C₁-C₂₀ haloalkoxy, hydroxy(C₁-C₂ alkyl), alkoxy(C₁-C₂₀ alkyl), —NH₂, —NH(C₁-C₂₀ alkyl), —N(C₁-C₂₀ alkyl)₂, —CONH₂, —CON H(C₁-C₂₀ alkyl), —CON(C₁-C₂₀ alkyl)₂, —NHCO(C₁-C₂₀ alkyl), —NHCO(C₁-C₂₀ alkoxy), —N(C₁-C₂₀ alkyl)CO(C₁-C₂₀ alkyl), —CO₂H, —CO₂(C₁-C₂₀ alkyl), —OCO(C₁-C₂₀ alkyl), —CO₂(aryl), —S(O)₀₋₂—(C₁-C₂₀ alkyl), —S(O)₀₋₂-aryl, —S(O)₀₋₂-heteroaryl, —P(O)(OH)₂, —P(O)(C₁-C₂₀ alkoxy)₂, —P(O)(aryloxy)₂, cycloalkyl, cycloalkyl(C₁-C₂₀ alkyl), aryl, aryl(C₁-C₂₀ alkyl), heteroaryl, heteroaryl(C₁-C₂₀ alkyl), heterocyclyl, and heterocyclyl(C₁-C₂₀ alkyl), or two R⁴ groups on the same non-aromatic atom form an oxo;
R¹ is C₁-C₂₀ alkyl, C₂-C₂₀ alkenyl, C₂-C₂₀ alkynyl optionally substituted with —Si(C₁-C₆alkyl)₃, C₄-C₁₀ haloalkyl, —CO₂(C₁-C₂₀ alkyl), —S(O)₀₋₂—(C₁-C₂₀ alkyl), —S(O)₀₋₂-aryl, —S(O)₀₋₂-heteroaryl, —P(O)(OH)₂, —P(O)(C₁-C₂₀ alkoxy)₂, —P(O)(aryloxy)₂, aryl, aryl(C₁-C₂₀ alkyl), heteroaryl, or heteroaryl(C₁-C₂₀ alkyl), wherein each of which is independently optionally substituted with one or more R⁵;
R² is C₁-C₂₀ alkyl, C₂-C₂₀ alkenyl, C₂-C₂₀ alkynyl optionally substituted with —Si(C₁-C₆ alkyl)₃, C₄-C₁₀ haloalkyl, —CO₂(C₁-C₂₀ alkyl), —S(O)₀₋₂—(C₁-C₂₀ alkyl), —S(O)₀₋₂-aryl, —S(O)₀₋₂-heteroaryl, —P(O)(OH)₂, —P(O)(C₁-C₂₀ alkoxy)₂, —P(O)(aryloxy)₂, aryl, aryl(C₁-C₂₀ alkyl), heteroaryl, or heteroaryl(C₁-C₂₀ alkyl), wherein each of which is independently optionally substituted with one or more R⁵;
wherein each R⁵ and R⁶ are independently selected from the group consisting of halogen, —NO₂, —CON, C₁₋₂₀ alkyl, C₂-C₂₀ alkenyl, C₂-C₂₀ alkynyl optionally substituted with —Si(C₁-C₆ alkyl)₃, C₁-C₂₀ haloalkyl, —OH, C₁-C₂₀ alkoxy, C₁-C₂₀ haloalkoxy, hydroxy(C₁-C₂₀ alkyl), alkoxy(C₁-C₂₀ alkyl), —NH₂, —NH(C₁-C₂₀ alkyl), —N(C₁-C₂₀ alkyl)₂, —CONH₂, —CON H(C₁-C₂₀ alkyl), —CON(C₁-C₂₀ alkyl)₂, —NHCO(C₁-C₂₀ alkyl), —N(C₁-C₂₀ alkyl)CO(C₁-C₂₀ alkyl), —CO₂H, —CO₂(C₁-C₂₀ alkyl), —OCO(C₁-C₂₀ alkyl), —CO₂(aryl), —S(O)₀₋₂—(C₁-C₂₀ alkyl), —S(O)₀₋₂-aryl, —S(O)₀₋₂-heteroaryl, —P(O)(OH)₂, —P(O)(C₁-C₂₀ alkoxy)₂, —P(O)(aryloxy)₂, cycloalkyl, cycloalkyl(C₁-C₂₀ alkyl), aryl, aryl(C₁-C₂₀ alkyl), heteroaryl, heteroaryl(C₁-C₂₀ alkyl), heterocyclyl, and heterocyclyl(C₁-C₂₀ alkyl), or two R⁵ groups on the same non-aromatic atom form an oxo, or two R⁶ groups on the same non-aromatic atom form an oxo; and
R³ is 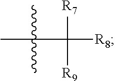
R⁷ is hydrogen, C₁-C₂₀ alkyl or aryl, wherein each alkyl or aryl moiety is optionally substituted with one or more R¹¹;
R⁸ is C₁-C₂₀ alkyl, aryl, —CO₂R¹⁰, or —CON(R¹⁰)₂; and
R⁹ is aryl, aryl(C₁-C₂ alkyl), heteroaryl, heteroaryl(C₁-C₂₀ alkyl), —CO₂R¹⁰, —CON(R¹)₂, or —CN, wherein each alkyl, aryl, or heteroaryl moiety is optionally substituted with one or more R¹¹; and
wherein each R¹⁰ is independently selected from the group consisting of hydrogen, C₁-C₂₀ alkyl, or aryl, wherein each alkyl or aryl moiety is optionally substituted with one or more R¹¹;
wherein each R¹¹ is independently selected from the group consisting of halogen, —NO₂, —ON, C₁-C₂₀ alkyl, C₂-C₂₀ alkenyl, C₂-C₂₀ alkynyl optionally substituted with —Si(C₁-C₂₀ alkyl)₃, C₁-C₂₀ haloalkyl, —OH, C₁-C₂₀ alkoxy, C₁-C₂₀ haloalkoxy, hydroxy(C₁-C₂₀ alkyl), alkoxy(C₁-C₂₀ alkyl), —NH₂, —NH(C₁-C₂₀ alkyl), —N(C₁-C₂₀ alkyl)₂, —CONH₂, —CONH(C₁-C₂₀ alkyl), —CON(C₁-C₂₀ alkyl)₂, —NHCO(C₁-C₂₀ alkyl), —N(C₁-C₂₀ alkyl)CO(C₁-C₂₀ alkyl), amino(C₁-C₂₀ alkyl), —CO₂H, —CO₂(C₁-C₂₀ alkyl), —OCO(C₁-C₂₀ alkyl), —CO₂(aryl), —S(O)₀₋₂—(C₁-C₂₀ alkyl), —S(O)₀₋₂-aryl, and —S(O)₀₋₂-heteroaryl, or two R¹¹ that are on non-aromatic atom form an oxo;
or R³ is a polymeric group resulting from polymerization of one or more of vinyl-containing monomers.

2. A compound according to claim 1, where R¹ is C₁-C₂₀ alkyl or aryl, each of which is optionally substituted with one or more R⁵.

3. A compound according to claim 2, where R¹ is aryl optionally substituted with one or more R⁵.

4. A compound according to claim 3, wherein R¹ is phenyl, methoxyphenyl, nitrophenyl, cyanophenyl, methylphenyl, isopropylphenyl or trimethylphenyl.

5. A compound according to claim 1, wherein R² is aryl optionally substituted with one or more R⁶.

6. A compound according to claim 1, wherein A is aryl optionally substituted with one or more R⁴.

7. A compound according to claim 1, wherein A is heteroaryl optionally substituted with one or more R⁴.

8. A compound according to claim 1, wherein R⁷ is C₁-C₂₀ alkyl, or hydrogen, and R⁸ is C₁-C₂₀ alkyl.

9. A compound according claim 8, wherein R⁹ is —CO₂R¹⁰, or —CON(R¹⁰)₂.

10. A compound according claim 8, wherein R⁹ is aryl optionally substituted with one or more R¹¹.

11. A compound according to claim 1, wherein R³ is a polymeric group resulting from polymerization of one or more of vinyl-containing monomers.

12. A compound according to claim 11, wherein R³ is a polymer resulting from polymerization of one or more of optionally substituted styrenes, optionally substituted alkylacrylates, optionally substituted alkylmethacrylates, acrylonitrile, methacrylonitrile, acrylamide, methacrylamide, isoprene, butadiene, ethylene, vinylacetate, vinyl ethers, and their combinations.

13. A compound of claim 1, which is:
1,3-diphenyl-4-(1-phenylethyl)-1,4-dihydrobenzo[e][1,2,4]triazine;
3-(4-methoxyphenyl)-1-phenyl-4-(1-phenylethyl)-1,4-dihydrobenzo[e][1,2,4]triazine;
1,3-diphenyl-4-(1-phenylethyl)-1,4-dihydrobenzo[e][1,2,4]triazine-7-carbonitrile;
4-(1-phenyl-4-(1-phenylethyl)-1,4-dihydrobenzo[e][1,2,4]triazin-3-yl)benzonitrile;
2,4-diphenyl-1-(1-phenylethyl)-1,4-dihydro-[1,2,4]triazino[6,5-h]quinolone;
5-methyl-1,3-diphenyl-4-(1-phenylethyl)-1,4-dihydrobenzo[e][1,2,4]triazine;
3-(4-nitrophenyl)-1-phenyl-4-(1-phenylethyl)-1,4-dihydrobenzo[e][1,2,4]triazine;
2,4-diphenyl-1-(1-phenylethyl)-1,4-dihydronaphtho[1,2-e][1,2,4]triazine;
ethyl 2-(1,3-diphenylbenzo[e][1,2,4]triazin-4(1H)-yl)propanoate;
5-ethyl-1,3-diphenyl-4-(1-phenylethyl)-1,4-dihydrobenzo[e][1,2,4]triazine;
5-tert-butyl-1,3-diphenyl-4-(1-phenylethyl)-1,4-dihydrobenzo[e][1,2,4]triazine;
ethyl 2-(2,4-diphenyl[1,2,4]triazino[6,5-h]quinolin-1 (4H)-yl)propanoate;
3-(naphthalen-1-yl)-1-phenyl-4-(1-phenylethyl)-1,4-dihydrobenzo[e][1,2,4]triazine;
3-mesityl-1-phenyl-4-(1-phenylethyl)-1,4-dihydrobenzo[e][1,2,4]triazine;
5-tert-butyl-3-mesityl-1-phenyl-4-(1-phenylethyl)-1,4-dihydrobenzo[e][1,2,4]triazine;
5-isopropyl-1,3-diphenyl-4-(1-phenylethyl)-1,4-dihydrobenzo[e][1,2,4]triazine;
7,9-diphenyl-10-(1-phenylethyl)-3,5,7,10-tetrahydropyreno[1,2-e][1,2,4]triazine;
(4-(1-(1,3-diphenylbenzo[e][1,2,4]triazin-4(1H)-yl)ethyl)phenyl)methanol; or
ethyl 2-(1,3-diphenylbenzo[e][1,2,4]triazin-4(1H)-yl)-2-methylpropanoate.

14. A method for polymerizing one or more vinyl-containing monomers comprising contacting one or more vinyl-containing monomers with one or more compounds according to claim 1.

15. A method according to claim 14, wherein the polymerizing results in a polymer having a polydispersity index of less than about 1.5.

16. A compound according to claim 3, wherein R² is aryl optionally substituted with one or more R⁶.

17. A compound according to claim 3, wherein A is aryl optionally substituted with one or more R⁴.

18. A compound according to claim 3, wherein A is heteroaryl optionally substituted with one or more R⁴.

19. A compound according to claim 3, wherein R⁷ is C₁-C₂₀ alkyl, or hydrogen, and R⁸ is C₁-C₂₀ alkyl.

20. A compound according claim 19, wherein R⁹ is —CO₂R¹⁰, or —CON(R¹⁰)₂.
